SEQUENCE LISTING (2 sequences)

SEQ ID NO: 1 - protein, 1212 aa, Human
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 2 - DNA, 6891 bp, Human
ggtggcctctgtggccgtccaggctagcggcggcccgcaggcggcggggagaaagactctctcacctggtcttgcggctgtggccaccgccggccaggggtgtggagggcgtgctgccggagacgtccgccgggctctgcagttccgccgggggtcgggcagctatggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccggctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagttgaggaagaggatggcaagactgcaactcaaccactgttgaaaaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataaatgttctatacagtggacagccctccagaatggtacttcagtgcctagtgtagtaactgaaatcttcaatgacacattaacatcacaatggcgaatggtgacttttctttcacgatttcattaatttgaaagcacacaggaaagttgctccattgataacgtgtatggagacttcggttttagtcaattccatatctcaatcttaatggtgattcttctctgttgaactgaagtttgtgagagtagttttcctttgctacttgaatagcaataaaagcgtgttaactttttgattgatgaaagaagtacaaaaagcctttagccttgaggtgccttctgaaattaaccaaatttcatccatatatcctcttttataaacttatagaatgtcaaactttgccttcaactgtttttatttctagtctcttccactttaaaacaaaatgaacactgcttgtcttcttccattgaccatttagtgttgagtactgtatgtgttttgttaattctataaaggtatctgttagatattaaaggtgagaattagggcaggttaatcaaaaatggggaaggggaaatggtaaccaaaaagtaaccccatggtaaggtttatatgagtatatgtgaatatagagctaggaaaaaaagcccccccaaatacctttttaacccctctgattggctattattactatatttattattatttattgaaaccttagggaagattgaagattcatcccatacttctatataccatgcttaaaaatcacgtcattctttaaacaaaaatactcaagatcatttatatttatttggagagaaaactgtcctaatttagaatttccctcaaatctgagggacttttaagaaatgctaacagatttttctggaggaaatttagacaaaacaatgtcatttagtagaatatttcagtatttaagtggaatttcagtatactgtactatcctttataagtcattaaaataatgtttcatcaaatggttaaatggaccactggtttcttagagaaatgtttttaggcttaattcattcaattgtcaagtacacttagtcttaatacactcaggtttgaacagattattctgaatattaaaatttaatccattcttaatattttaaaacttttgttaagaaaaactgccagtttgtgcttttgaaatgtctgttttgacatcatagtctagtaaaattttgacagtgcatatgtactgttactaaaagctttatatgaaattattaatgtgaagtttttcatttataattcaaggaaggatttcctgaaaacatttcaagggatttatgtctacatatttgtgtgtgtgtgtgtatatatatgtaatatgcatacacagatgcatatgtgtatatataatgaaatttatgttgctggtattttgcattttaaagtgatcaagattcattaggcaaactttggtttaagtaaacatatgttcaaaatcagattaacagatacaggtttcatagagaacaaaggtgatcatttgaagggcatgctgtaatttcacacaattttccagttcaaaaatggagaatacttcgcctaaaatactgttaagtgggttaattgatacaagtttctgtggtggaaaatttatgcaggttttcacgaatccttttttttttttttttttttttttgagacggagtcttgctctgttgccacgctggaatgcagtaacgtgatcttggctcactgcgacctccacctccccagttcaagcgattctcctgcctcagcctccctagtagctgggactacgggtgcacgccaccatgcccagctaatttttgtattttgagtagagacagggtttcaccgtgttggctaggatggtgtctatctcttgaccttgtgatccacccgcctcagcctcccagagtgctgggattacaggtgcgagccactgcgcctggctggttttcatgaatcttgatagacatctataacgttattattttcagtggtgtgcagcatttttgcttcatgagtatgacctaggtatagagatctgataacttgaattcagaatattaagaaaatgaagtaactgattttctaaaaaaaaaaaaaaaaaaaatttctacattataactcacagcattgttccattgcaggttttgcaatgtttgggggtaaagacagtagaaatattattcagtaaacaataatgtgtgaacttttaagatggataatagggcatggactgagtgctgctatcttgaaatgtgcacaggtacacttaccttttttttttttttttttaagtttttcccattcaggaaaacaacattgtgatctgtactacaggaaccaaatgtcatgcgtcatacatgtgggtataaagtacataaaatatatctaactattcataatgtggggtgggtaatactgtctgtgaaataatgtaagaagcttttcacttaaaaaaaatgcattactttcacttaacactagacaccaggtcgaaaattttcaaggttatagtacttatttcaacaattcttagagatgctagctagtgttgaagctaaaaatagctttatttatgctgaattgtgatttttttatgccaaattttttttagttctaatcattgatgatagcttggaaataaataattatgccatggcatttgacagttcattattcctataagaattaaattgagtttagagagaatggtggtgttgagctgattattaacagttactgaaatcaaatatttatttgttacattattccatttgtattttaggtttccttttacattctttttatatgcattctgacattacatattttttaagactatggaaataatttaaagatttaagctctggtggatgattatctgctaagtaagtctgaaaatgtaatattttgataatactgtaatatacctgtcacacaaatgcttttctaatgttttaaccttgagtattgcagttgctgctttgtacagaggttactgcaataaaggaagtggattcattaaacctatttaatgtccaaaaaaaaaaaaaaaaaaaaaaaaaaaaaaaa

(19) United States
(12) Patent Application Publication
(10) Pub. No.: US 2006/0025387 A1
(43) Pub. Date: Feb. 2, 2006

(54) Compositions and methods for the treatment of disorders of the central and peripheral nervous systems

(75) Inventor: Daryl W. Hochman, Bahama, NC (US)

Correspondence Address: SPECKMAN LAW GROUP PLLC, 1501 WESTERN AVE, SEATTLE, WA, 98101, US

(73) Assignee: CYTOSCAN SCIENCES LLC, Seattle, WA (US)

(21) Appl. No.: 11/130,945

(22) Filed: May 17, 2005

Related U.S. Application Data
(63) Continuation-in-part of application No. 11/101,000, filed on Apr. 7, 2005.
Application No. 11/101,000 is a continuation-in-part of application No. 10/056,528, filed on Jan. 23, 2002.
Application No. 11/101,000 is a continuation-in-part of application No. 09/470,637, filed on Dec. 22, 1999, now Pat. No. 6,495,601.
(60) Provisional application No. 60/263,830, filed on Jan. 23, 2001.
(60) Provisional application No. 60/113,620, filed on Dec. 23, 1998.

(51) Int. Cl. A61K 31/5513; A61K 31/55; A61K 31/549; A61K 31/513; A61K 31/485; A61K 31/48; A61K 31/4166; A61K 31/19

(52) U.S. Cl. 514/165; 514/269; 514/223.5; 514/282; 514/221; 514/557; 514/569; 514/288; 514/355; 514/171; 514/270; 514/217; 514/389; 514/561; 514/419

(57) ABSTRACT

The present invention relates to methods and compositions for treating disorders of the central and/or peripheral nervous system by administering agents that are effective in reducing the effective amount, inactivating, and/or inhibiting the activity of a Na⁺—K⁺—2CT (NKCC) cotransporter. In certain embodiments, the Na⁺—K⁺—2Cl⁻ co-transporter is NKCC1.

### REFERENCE TO RELATED APPLICATIONS

This application is a continuation-in-part of U.S. patent application Ser. No. 11/101,000, filed Apr. 7, 2005, which is a continuation-in-part of U.S. patent application Ser. No. 10/056,528, filed Jan. 23, 2002, which claims priority under 35 U.S.C. §119(e) to U.S. patent application Ser. No. 60/263,830, filed Jan. 23, 2001, and is a continuation-in-part of U.S. patent application Ser. No. 09/470,637, filed Dec. 22, 1999, now U.S. Pat. No. 6,495,601, which claims priority under 35 U.S.C. §119(e) to U.S. patent application Ser. No. 60/113,620, filed Dec. 23, 1998.

### TECHNICAL FIELD OF THE INVENTION

The present invention relates to methods and compositions for treating selected conditions of the central and peripheral nervous systems employing non-synaptic mechanisms. More specifically, the present invention relates to methods and compositions for treating seizures and seizure disorders, epilepsy, status epilepticus, migraine headache, cortical spreading depression, intracranial hypertension, neuropsychiatric disorders, addictive or compulsive disorders, neuropathic pain, central nervous system edema; for treating or preventing the pathophysiological effects of toxic agents such as ethanol and certain infectious agents; for treating the pathophysiological effects of head trauma, stroke, ischemia and hypoxia; and for improving certain brain functions, such as cognition, learning and memory by administering agents that modulate expression and/or activity of sodium-potassium-chloride co-transporters.

### BACKGROUND OF THE INVENTION

Conventional treatments for neuronal disorders, such as seizure disorders, epilepsy and the like, target synaptic mechanisms that affect excitatory pathways, for example by modulating the release or activity of neurotransmitters or inhibitors. Conventional treatment agents and regimen for seizure disorders diminish neuronal excitability and inhibit synaptic firing. One serious drawback of this approach is that while seizures are generally localized, the treatment diminishes neuronal activity indiscriminately. For this reason, there are serious side effects and repeated use of conventional medications may result in unintended deficiencies in normal and desirable brain functions, such as cognition, learning and memory. More detailed information concerning particular disorders of interest is provided below.

Epilepsy

Epilepsy is characterized by abnormal discharges of cerebral neurons and is typically manifested as various types of seizures. Epileptiform activity is identified with spontaneously occurring synchronized discharges of neuronal populations that can be measured using electrophysiological techniques. Epilepsy is one of the most common neurological disorders, affecting about 1% of the population. There are various forms of epilepsy, including idiopathic, symptomatic and cryptogenic. Genetic predisposition is thought to be the predominant etiologic factor in idiopathic epilepsy. Symptomatic epilepsy usually develops as a result of a structural abnormality in the brain.

Status epilepticus is a particularly severe form of seizure, which is manifested as multiple seizures that persist for a significant length of time, or serial seizures without any recovery of consciousness between seizures. The overall mortality rate among adults with status epilepticus is approximately 20 percent. Patients who have a first episode are at substantial risk for future episodes and for the development of chronic epilepsy. The frequency of status epilepticus in the United States is approximately 150,000 cases per year, with approximately 55,000 deaths being associated with status epilepticus annually. Acute processes that are associated with status epilepticus include intractable epilepsy, metabolic disturbances (e.g. electrolyte abnormalities, renal failure and sepsis), central nervous system infection (meningitis or encephalitis), stroke, degenerative diseases, head trauma, drug toxicity and hypoxia. The fundamental pathophysiology of status epilepticus involves a failure of mechanisms that normally abort an isolated seizure. This failure can arise from abnormally persistent, excessive excitation or ineffective recruitment of inhibition. Studies have shown that excessive activation of excitatory amino acid receptors can cause prolonged seizures and suggest that excitatory amino acids may play a causative role. Status epilepticus can also be caused by penicillin and related compounds that antagonize the effects of γ-aminobutyric acid (GABA), the primary inhibitory neurotransmitter in the brain.

One early diagnostic procedure for epilepsy involved the oral administration of large quantities of water together with injections of vasopressin to prevent the accompanying diuresis. This procedure was found to induce seizures in epileptic patients, but rarely in non-epileptic individuals (Garland et al., Lancet, 2:566, 1943). Status epilepticus can be blocked in kainic acid-treated rats by intravenous injection of mannitol (Baran et al., Neuroscience, 21:679, 1987). This effect is similar to that achieved by intravenous injection of urea in human patients (Carter, Epilepsia, 3:198, 1962). The treatment in each of these cases increases the osmolarity of the blood and extracellular fluid, resulting in water efflux from the cells and an increase in extracellular space in the brain. Acetazolamide (ACZ), another diuretic with a different mechanism of action (inhibition of carbonic anhydrase), has been studied experimentally as an anticonvulsant (White et al., Advance Neurol., 44:695, 1986; and Guillaume et al., Epilepsia, 32:10, 1991) and used clinically on a limited basis (Tanimukai et al., Biochem. Pharm., 14:961, 1965; and Forsythe et al., Develop. Med. Child Neurol., 23:761, 1981). Although its mechanism of anticonvulsant action has not been determined, ACZ does have a clear effect on the cerebral extracellular space.

Traditional anti-epileptic drugs exert their principal effect through one of three mechanisms: (a) inhibition of repetitive, high-frequency neuronal firing by blocking voltage-dependent sodium channels; (b) potentiation of γ-aminobutyric acid (GABA)-mediated postsynaptic inhibition; and (c) blockade of T-type calcium channels. Phenytoin and carbamazepine are examples of sodium channel antagonists which exert their effect at the cellular level by reducing or eliminating sustained high-frequency neuronal depolarization while not appreciably affecting regular firing rates of neurons. Barbiturates, such as phenobarbital and benzodiazepines, act by enhancing GABA-mediated synaptic inhibition. Both classes of compounds increase the hyperpolarization of the postsynaptic membrane, resulting in increased inhibition. Ethosuximide and valporate are examples of drugs that decrease calcium entry into neurons through T-type voltage-dependent calcium channels.

Current anti-epileptic drug therapies exert their pharmacological effects on all brain cells, regardless of their involvement in seizure activity. Common side effects are over-sedation, dizziness, loss of memory and liver damage. Furthermore, 20-30% of epilepsy patients are refractory to current therapy.

Migraine

Migraine headaches afflict 10-20% of the U.S. population, with an estimated loss of 64 million workdays annually. Migraine headache is characterized by pulsating head pain that is episodic, unilateral or bilateral, lasting from 4 to 72 hours and often associated with nausea, vomiting and hypersensitivity to light and/or sound. When accompanied by premonitory symptoms, such as visual, sensory, speech or motor symptoms, the headache is referred to as “migraine with aura,” formerly known as classic migraine. When not accompanied by such symptoms, the headache is referred to as “migraine without aura,” formerly known as common migraine. Both types evidence a strong genetic component, and both are three times more common in women than men. The precise etiology of migraine has yet to be determined. It has been theorized that persons prone to migraine have a reduced threshold for neuronal excitability, possibly due to reduced activity of the inhibitory neurotransmitter γ-aminobutyric acid (GABA). GABA normally inhibits the activity of the neurotransmitters serotonin (5-HT) and glutamate, both of which appear to be involved in migraine attacks. The excitatory neurotransmitter glutamate is implicated in an electrical phenomenon called cortical spreading depression, which can initiate a migraine attack, while serotonin is implicated in vascular changes that occur as the migraine progresses.

It has been suggested that cortical spreading depression (CSD) underlies migraine visual aura. CSD is characterized by a short burst of intense depolarization in the occipital cortex, followed by a wave of neuronal silence and diminished evoked potentials that advance anteriorly across the surface of the cerebral cortex. Enhanced excitability of the occipital-cortex neurons has been proposed as the basis for CSD. The visual cortex may have a lower threshold for excitability and therefore is most prone to CSD. It has been suggested that mitochondrial disorders, magnesium deficiency and abnormality of presynaptic calcium channels may be responsible for neuronal hyperexcitability (Welch, Pathogenesis of Migraine, Seminars in Neurobiol., 17:4, 1997). During a spreading depression event, profound ionic perturbations occur, which include interstitial acidification, extracellular potassium accumulation and redistribution of sodium and chloride ions to intracellular compartments. In addition, prolonged glial swelling occurs as a homeostatic response to altered ionic extracellular fluid composition, and interstitial neurotransmitter and fatty acid accumulation. Studies have shown that furosemide inhibits regenerative cortical spreading depression in anaesthetized cats (Read et al, Cephalagia, 17:826, 1997).

A study of eighty-five patients with refractory transformed migraine type of chronic daily headache (CDH) concluded that acute headache exacerbations responded to specific anti-migraine agents such as ergotamine, dihydroergotamine (DHE) and sumatriptan, and that addition of agents such as acetazolamide and furosemide, after diagnosis of increased intracranial pressure, resulted in better control of symptoms (Mathew et al. Neurology 46:1226-1230, May 1996). The authors note that these results suggest a possible link between migraine and idiopathic intracranial hypertension that needs further research. It has also been reported that furosemide appeared to abort prolonged visual auras in two migraine patients. The author speculated that furosemide may act to inhibit CSD activity (Rozen, Neurology, 55:732-3, 2000).

Drug therapy is tailored to the severity and frequency of migraine headaches. For occasional attacks, abortive treatment may be indicated, but for attacks occurring two or more times per month, or when attacks greatly impact the patient's daily life, prophylactic therapy may be indicated. Serotonin receptor agonists, such as sumatriptan, have been prescribed for abortive therapy. These are thought to constrict dilated arteries of the brain, thereby alleviating the associated pain. Side effects associated with the use of serotonin receptor agonists include tingling, dizziness, warm-hot sensations and injection-site reactions. Intravenous administration is contraindicated due to the potential for coronary vasospasms.

Drugs used for prophylactic treatment of migraine include andrenergic beta-blockers such as propranolol, calcium channel blockers, and low-dose anti-epileptic drugs. In particular, anti-epileptic drugs that increase brain levels of GABA, either by increasing GABA synthesis or reducing its breakdown, appear to be effective in preventing migraine in certain individuals. In some patients, tricyclic analgesics, such as amitriptline, can be effective. NMDA receptor antagonists, which act at one of the glutamate receptor subtypes in the brain, inhibit CSD. Drugs or substances currently believed to function as weak NMDA receptor antagonists include dextromethoraphan, magnesium and ketamine. Intravenous magnesium has been successfully used to abort migraine attacks.

Neurotoxicity

A variety of chemical and biological agents, as well as some infectious agents, have neurotoxic effects. A common example is the pathophysiological effect of acute ethanol ingestion. Episodic ethanol intoxications and withdrawals, characteristic of binge alcoholism, result in brain damage. Animal models designed to mimic the effects of alcohol in the human have demonstrated that a single dose of ethanol given for 5-10 successive days results in neurodegeneration in the entorhinal cortex, dentate gyrus and olfactory bulbs, accompanied by cerebrocortical edema and electrolyte (Na⁺ and K⁺) accumulation. As with other neurodegenerative conditions, research has focused primarily on synaptically based excitotoxic events involving excessive glutamatergic activity, increased intracellular calcium and decreased γ-aminobutyric acid. Co-treatment of brain damage induced by episodic alcohol exposure with an NMDA receptor antagonist, non-NMDA receptor and Ca²⁺ channel antagonists with furosemide reduces alcohol-dependent cerebrocortical damage by 75-85%, while preventing brain hydration and electrolyte elevations (Collins et al, FASEB, vol. 12, February 1998). The authors suggested that furosemide and related agents might be useful as neuroprotective agents in alcohol abuse.

Cognition, learning and memory

The cognitive abilities of mammals are thought to be dependent on cortical processing. It has generally been accepted that the most relevant parameters for describing and understanding cortical function are the spatio-temporal patterns of activity. In particular, long-term potentiation and long-term depression have been implicated in memory and learning and may play a role in cognition. Oscillatory and synchronized activities in the brains of mammals have been correlated with distinct behavioral states.

Synchronization of spontaneous neuronal firing activity is thought to be an important feature of a number of normal and pathophysiological processes in the central nervous system. Examples include synchronized oscillations of population activity such as gamma rhythms in the neocortex, which are thought to be involved in cognition (Singer and Gray, Annu. Rev. Neurosci., 18:855-86, 1995), and theta rhythm in hippocampus, which is thought to play roles in spatial memory and in the induction of synaptic plasticity (Heurta and Lisman, Neuron. 15:1053-63, 1995; Heurta and Lisman, J. Neurophysiol. 75:877-84, 1996; O'Keefe, Curr. Opin. Neurobiol., 3:917-24, 1993). To date, most research on the processes underlying the generation and maintenance of spontaneous synchronized activity has focused on synaptic mechanisms. However, there is evidence that nonsynaptic mechanisms may also play important roles in the modulation of synchronization in normal and pathological activities in the central nervous system.

Addictive Disorders

Addictive and/or compulsive disorders, such as eating disorders (including obesity), addiction to narcotics, alcoholism, and smoking are a major public health problem that impacts society on multiple levels. It has been estimated that substance abuse costs the US more than $484 billion per year. Current strategies for the treatment of additive disorders include psychological counseling and support, use of therapeutic agents or a combination of both. A variety of agents known to affect the central nervous system have been used in various contexts to treat a number of indications related directly or indirectly to addictive behaviors. For example, the combination of phentermine and fenfluramine was used for many years to exert an anorectic effect to treat obesity.

Topiramate is an anti-convulsant that was originally developed as an anti-diabetic agent and is approved for use in the treatment of epileptic seizures in adults and children. It is a GABA-receptor agonist and has sodium channel-blocking activity. Studies on the effectiveness of topiramate in treating alcohol dependence demonstrated that oral administration of topiramate led to a decrease in heavy drinking days and alcohol craving, with a concurrent increase in abstinent days and improved liver functions (Johnson et al. Lancet, 361:1677-85, 2003). Topiramate has also been shown to be effective in treating binge eating disorder associated with obesity (McElroy et al. Am. J. Psychiatry 160:255-261, 2003; McElroy et al. J. Clin. Psychiatry 65:1463-9, 2004), and bipolar disorder (Suppes, J. Clin. Psychopharmacol. 22:599-609, 2002). More recently, it has been suggested that topiramate may be an effective treatment for obesity.

Neuropathic Pain

Neuropathic pain and nociceptive pain differ in their etiology, pathophysiology, diagnosis and treatment. Nociceptive pain occurs in response to the activation of a specific subset of peripheral sensory neurons, the nociceptors. It is generally acute (with the exception of arthritic pain), self-limiting and serves a protective biological function by acting as a warning of on-going tissue damage. It is typically well localized and often has an aching or throbbing quality. Examples of nociceptive pain include post-operative pain, sprains, bone fractures, burns, bumps, bruises, inflammation (from an infection or arthritic disorder), obstructions and myofascial pain. Nociceptive pain can usually be treated with opioids and non-steroidal anti-inflammatory drugs (NSAIDS).

Neuropathic pain is a common type of chronic, non-malignant, pain, which is the result of an injury or malfunction in the peripheral or central nervous system and serves no protective biological function. It is estimated to affect more than 1.6 million people in the U.S. population. Neuropathic pain has many different etiologies, and may occur, for example, due to trauma, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs.

In contrast to nociceptive pain, neuropathic pain is frequently described as “burning”, “electric”, “tingling” or “shooting” in nature. It is often characterized by chronic allodynia (defined as pain resulting from a stimulus that does not ordinarily elicit a painful response, such as light touch) and hyperalgesia (defined as an increased sensitivity to a normally painful stimulus), and may persist for months or years beyond the apparent healing of any damaged tissues.

Neuropathic pain is difficult to treat. Analgesic drugs that are effective against normal pain (e.g., opioid narcotics and non-steroidal anti-inflammatory drugs) are rarely effective against neuropathic pain. Similarly, drugs that have activity in neuropathic pain are not usually effective against nociceptive pain. The standard drugs that have been used to treat neuropathic pain appear to often act selectively to relieve certain symptoms but not others in a given patient (for example, relief of allodynia, but not hyperalgesia). For this reason, it has been suggested that successful therapy may require the use of multiple different combinations of drugs and individualized therapy (see, for example, Bennett, Hosp. Pract. (Off Ed). 33:95-98, 1998). Treatment agents typically employed in the management of neuropathic pain include tricylic antidepressants (for example, amitriptyline, imipramine, desimipramine and clomipramine), systemic local anesthetics, and anti-convulsants (such as phenytoin, carbamazepine, valproic acid, clonazepam and gabapentin).

Many anti-convulsants originally developed for the treatment of epilepsy and other seizure disorders have found application in the treatment of non-epileptic conditions, including neuropathic pain, mood disorders (such as bipolar affective disorder), and schizophrenia (for a review of the use of anti-epileptic drugs in the treatment of non-epileptic conditions, see Rogawski and Loscher, Nat. Medicine, 10:685-692, 2004). It has thus been suggested that epilepsy, neuropathic pain and affective disorders have a common pathophysiological mechanism (Rogawski & Loscher, ibid; Ruscheweyh & Sandkuhler, Pain 105:327-338, 2003), namely a pathological increase in neuronal excitability, with a corresponding inappropriately high frequency of spontaneous firing of neurons. However, only some, and not all, antiepileptic drugs are effective in treating neuropathic pain, and furthermore such antiepileptic drugs are only effective in certain subsets of patients with neuropathic pain (McCleane, Expert. Opin. Pharmacother. 5:1299-1312, 2004).

As discussed above, epilepsy is characterized by abnormal discharges of cerebral neurons and is typically manifested as various types of seizures, with epileptiform activity being identified with spontaneously occurring synchronized discharges of neuronal populations that can be measured using electrophysiological techniques. This synchronized activity, which distinguishes epileptiform from non-epileptiform activity, is referred to as “hypersynchronization” because it describes the state in which individual neurons become increasingly likely to discharge in a time-locked manner with one another. Hypersynchronized activity is typically induced in experimental models of epilepsy by either increasing excitatory or decreasing inhibitory synaptic currents, and it was therefore assumed that hyperexcitability per se was the defining feature involved in the generation and maintenance of epileptiform activity. Similarly, neuropathic pain was believed to involve conversion of neurons involved in pain transmission from a state of normal sensitivity to one of hypersensitivity (Costigan & Woolf, Jnl. Pain 1:35-44, 2000). The focus on developing treatments for both epilepsy and neuropathic pain has thus been on suppressing neuronal hyperexcitability by either: (a) suppressing action potential generation; (b) increasing inhibitory synaptic transmission; or (c) decreasing excitatory synaptic transmission. However, it has been shown that hypersychronous epileptiform activity can be dissociated from hyperexcitability and that the cation chloride cotransport inhibitor furosemide reversibly blocked synchronized discharges without reducing hyperexcited synaptic responses (Hochman et al. Science 270:99-102, 1995).

Both abnormal expression of sodium channel genes (Waxman, Pain 6:S133-140, 1999; Waxman et al. Proc. Natl. Acad. Sci USA 96:7635-7639, 1999) and pacemaker channels (Chaplan et al. J. Neurosci. 23:1169-1178, 2003) are believed to play a role in the molecular basis of neuropathic pain.

The cation-chloride co-transporters (CCCs) are important regulators of neuronal chloride concentration that are believed to influence cell-to-cell communication, and various aspects of neuronal development, plasticity and trauma. The CCC gene family consists of three broad groups: Na⁺—Cl⁻ co-transporters (NCCs), K⁺—Cl⁻ co-transporters (KCCs) and Na⁺K⁺—2Cl⁻ co-transporters (NKCCs). Two NKCC isoforms have been identified: NKCC1 is found in a wide variety of secretory epithelia and non-epithelial cells, whereas NKCC2 is principally expressed in the kidney. For a review of NKCC1 structure, function and regulation see, Haas and Forbush, Annu. Rev. Physiol. 62:515-534, 2000. Randall et al. have identified two splice variants of the Slc12a2 gene that encodes NKCC1, referred to as NKCC1a and NKCC1b (Am. J. Physiol. 273 (Cell Physiol. 42):C1267-1277, 1997). The NKCC1 a gene has 27 exons, while the splice variant NKCC1b lacks exon 21. The NKCC1b splice variant is expressed primarily in the brain. NKCC1b is believed to be more than 10% more active than NKCC1a, although it is proportionally present in a much smaller amount in the brain than is NKCC1a. It has been suggested that differential splicing of the NKCC1 transcript may play a regulatory role in human tissues (Vibat et al. Anal. Biochem. 298:218-230, 2001). Na—K—Cl co-transport in all cell and tissues is inhibited by loop diuretics, including furosemide, bumetanide and benzmetanide.

Na—K—2Cl co-transporter knock-out mice have been shown to have impaired nociception phenotypes as well as abnormal gait and locomotion (Sung et al. Jnl. Neurosci. 20:7531-7538, 2000). Delpire and Mount have suggested that NKCC1 may be involved in pain perception (Ann. Rev. Physiol. 64:803-843, 2002). Laird et al. recently described studies demonstrating reduced stroking hyperalgesia in NKCC1 knock-out mice compared to wild-type and heterozygous mice (Neurosci. Letts. 361:200-203, 2004). However, in this acute pain model no difference in punctuate hyperalgesia was observed between the three groups of mice. Morales-Aza et al. have suggested that, in arthritis, altered expression of NKCC1 and the K—Cl co-transporter KCC2 may contribute to the control of spinal cord excitability and may thus represent therapeutic targets for the treatment of inflammatory pain (Neurobiol. Dis. 17:62-69, 2004). Granados-Soto et al. have described studies in rats in which formalin-induced nociception was reduced by administration of the NKCC inhibitors bumetanide, furosemide or piretanide (Pain 114:231-238, 2005). While the formalin-induced acute pain model is extensively used, it is believed to have little relevance to chronic pain conditions (Walker et al. Mol. Med. Today 5:319-321, 1999). Co-treatment of brain damage induced by episodic alcohol exposure with an NMDA receptor antagonist, non-NMDA receptor and Ca²⁺ channel antagonists together with furosemide has been shown to reduce alcohol-dependent cerebrocortical damage by 75-85%, while preventing brain hydration and electrolyte elevations (Collins et al, FASEB J., 12:221-230, 1998). The authors stated that the results suggest that furosemide and related agents might be useful as neuroprotective agents in alcohol abuse. Willis et al. have published studies indicating that nedocromil sodium, furosemide and bumetanide inhibit sensory nerve activation to reduce the itch and flare responses induced by histamine in human skin in vivo. Espinosa et al. and Ahmad et al. have previously suggested that furosemide might be useful in the treatment of certain types of epilepsy (Medicina Espanola 61:280-281, 1969; and Brit. J. Clin. Pharmacol. 3:621-625, 1976).

As with epilepsy, the focus of pharmacological intervention in many disorders of the central and peripheral nervous system, including neuropathic pain, has been on reducing neuronal hyperexcitability. Most agents currently used to treat such disorders target synaptic activity in excitatory pathways by, for example, modulating the release or activity of excitatory neurotransmitters, potentiating inhibitory pathways, blocking ion channels involved in impulse generation, and/or acting as membrane stabilizers. Conventional agents and therapeutic approaches for the treatment of central and peripheral nervous system disorders thus reduce neuronal excitability and inhibit synaptic firing. One serious drawback of these therapies is that they are nonselective and exert their actions on both normal and abnormal neuronal populations. This leads to negative and unintended side effects, which may affect normal CNS functions, such as cognition, learning and memory, and produce adverse physiological and psychological effects in the treated patient. Common side effects include over-sedation, dizziness, loss of memory and liver damage. There is therefore a continuing need for methods and compositions for treating central and peripheral nervous system disorders that disrupt hypersynchronized neuronal activity without diminishing the neuronal excitability and spontaneous synchronization required for normal functioning of the peripheral and central nervous systems.

Use of Diuretics in the treatment of non-CNS disorders

Individuals with disorders such as hypertension and congestive heart failure frequently take large doses of diuretics, including loop diuretics, which work by blocking the absorption of salt and fluid in the kidney tubules, leading to a profound increase in urine output (diuresis). While the resulting loss of water has a positive effect on disorders such as hypertension and congestive heart failure, this loss of water is not desirable in disorders such as epilepsy, migraine and neuropathic pain. In addition, the loss of water resulting from administration of diuretic compositions is accompanied by loss of electrolytes and vitamins which can lead to deficiencies in, for example, potassium, magnesium and thiamine (Zenuk et al., Can. J Clin. Pharmacol., 10:184-8, 2003; Schwinger and Erdmann, Methods Find. Exp. Clin. Pharmacol., 14:315-25, 1992; Ryan, Magnesium, 5:282-92, 1986; Cohen et al., Clin. Cardiol., 23:433-436, 2000). This depletion of electrolytes can have significant negative side effects. For example, depletion of potassium can lead to abnormal heart rhythms, weakness and confusion. U.S. Pat. No. 4,855,289 discloses the use of a compound having diuretic properties, a magnesium supplement and/or a potassium supplement in the treatment of hypertension and/or congestive heart failure.

Screening of Candidate Compounds and Evaluating Treatment Efficacy

Drug development programs rely on in vitro screening assays and subsequent testing in appropriate animal models to evaluate drug candidates prior to conducting clinical trials using human subjects. Screening methods currently used are generally difficult to scale up to provide the high throughput screening necessary to test the numerous candidate compounds generated by traditional and computational means. Moreover, studies involving cell culture systems and animal model responses may not accurately predict the responses and side effects observed during human clinical trials.

Conventional methods for assessing the effects of various agents or physiological activities on biological materials, in both in vitro and in vivo systems, are generally not highly sensitive or informative. For example, assessment of the effect of a physiological agent, such as a drug, on a population of cells or tissue grown in culture conventionally provides information relating to the effect of the agent on the cell or tissue population only at specific points in time. Additionally, current assessment techniques generally provide information relating to a single or a small number of parameters. Candidate agents are systematically tested for cytotoxicity, which may be determined as a function of concentration. A population of cells is treated and, at one or several time points following treatment, cell survival is measured. Cytotoxicity assays generally do not provide any information relating to the cause(s) or time course of cell death.

Similarly, agents are frequently evaluated based on their physiological effects, for example, on a particular metabolic function or metabolite. An agent is administered to a population of cells or a tissue sample, and the metabolic function or metabolite of interest is assayed to assess the effect of the agent. This type of assay provides useful information, but it does not provide information relating to the mechanism of action, the effect on other metabolites or metabolic functions, the time course of the physiological effect, general cell or tissue health, or the like.

U.S. Pat. Nos. 5,902,732 and 5,976,825 disclose methods for screening drug candidate compounds for anti-epileptic activity using glial cells in culture by osomotically shocking glial cells, introducing a drug candidate, and assessing whether the drug candidate is capable of abating changes in glial cell swelling. These patents also disclose a method for screening drug candidate compounds for activity to prevent or treat symptoms of Alzheimer's disease, or to prevent CNS damage resulting from ischemia, by adding a sensitization agent capable of inducing apoptosis and an osmotic stressing agent to CNS cells, adding the drug candidate, and assessing whether the drug candidate is capable of abating cell swelling. A method for determining the viability and health of living cells inside polymeric tissue implants is also disclosed, involving measuring dimensions of living cells inside the polymeric matrix, osmotically shocking the cells, and then assessing changes in cell swelling. Assessment of cell swelling activity is achieved by measuring intrinsic optical signals using an optical detection system. U.S. Pat. Nos. 6,096,510 and 6,319,682 disclose additional methods for screening drug candidate compounds.

### SUMMARY OF THE INVENTION

The treatment compositions and methods of the present invention are useful for treating and/or preventing conditions that are characterized by neuronal hypersynchrony. Such disorders include: addictive and compulsive disorders, such as eating disorders (including obesity and binge eating), alcoholism, addiction to narcotics and smoking; neuropathic pain; neuropsychiatric disorders, such as bipolar disorders, anxiety, panic attacks, depression, schizophrenia and post-traumatic stress syndrome; seizures and seizure disorders; epilepsy (including Status epilepticus); migraine headaches and other types of headaches; cortical spreading depression; intracranial hypertension; central nervous system edema; the pathophysiological effects of neurotoxic agents, such as ethanol and certain infectious agents; and the pathophysiological effects of head trauma, stroke, ischemia and hypoxia. Treatment compositions and methods of the present invention may also be employed to improve function in certain cortical tissue, such as in cortical centers of cognition, learning and memory. The inventive compositions and methods may be employed to reduce neuronal hypersynchrony associated with such conditions without suppressing neuronal excitability, thereby avoiding the unwanted side effects often associated with agents currently employed for the treatment of disorders of the central and peripheral nervous systems.

The methods and compositions disclosed herein generally involve non-synaptic mechanisms and modulate, generally reduce the synchronization of neuronal population activity. The synchronization of neuronal population activity is modulated by manipulating anionic concentrations and gradients in the central and/or peripheral nervous systems. More specifically, the inventive compositions are capable of reducing the effective amount, inactivating, and/or inhibiting the activity of a Na⁺—K⁺—2Cl⁻ (NKCC) co-transporter. Preferred treatment agents of the present invention exhibit a high degree of NKCC co-transporter antagonist activity in cells of the central and/or peripheral nervous system, e.g., glial cells, Schwann cells and/or neuronal cell populations, and exhibit a lesser degree of activity in renal cell populations. In one embodiment, the inventive compositions are capable of reducing the effective amount, inactivating, and/or inhibiting the activity of the co-transporter NKCC1. NKCC1 antagonists are preferred treatment agents for use in the inventive methods. NKCC co-transporter antagonists that may be usefully employed in the inventive treatment compositions include, but are not limited to, loop diuretics such as furosemide, bumetanide, ethacrynic acid, torsemide, azosemide, muzolimine, piretanide, tripamide and the like, as well as thiazide and thiazide-like diuretics, such as bendroflumethiazide, benzthiazide, chlorothiazide, hydrochlorothiazide, hydroflumethiazide, methylclothiazide, polythiazide, trichlormethiazide, chlorthalidone, indapamide, metolazone and quinethazone, together with analogs and functional derivatives of such components.

Other treatment agents that may be usefully employed in the inventive compositions and methods include, but are not limited to: antibodies, or antigen-binding fragments thereof, that specifically bind to NKCC1; soluble NKCC1 ligands; small molecule inhibitors of NKCC1; anti-sense oligonucleotides to NKCC1; NKCC1-specific small interfering RNA molecules (siRNA or RNAi); and engineered soluble NKCC1 molecules. Preferably, such antibodies, or antigen-binding fragments thereof, and small molecule inhibitors of NKCC1, specifically bind to the domains of NKCC1 involved in bumetanide binding, as described, for example, in Haas and Forbush II, Annu. Rev. Physiol. 62:515-534, 2000. The polypeptide sequence for human NKCC1 is provided in SEQ ID NO: 1, with the corresponding cDNA sequence being provided in SEQ ID NO: 2.

As the methods and treatment agents of the present invention employ “non-synaptic” mechanisms, little or no suppression of neuronal excitability occurs. More specifically, the inventive treatment agents cause little (less than a 1% change compared to pre-administration levels) or no suppression of action potential generation or excitatory synaptic transmission. In fact, a slight increase in neuronal excitability may occur upon administration of certain of the inventive treatment agents. This is in marked contrast to conventional anti-epileptic drugs currently used in the treatment of many central and peripheral nervous system disorders, which do suppress neuronal excitability. The methods and treatment agents of the present invention affect the synchronization, or relative synchrony, of neuronal population activity. Preferred methods and treatment agents modulate the extracellular anionic chloride concentration and/or the gradients in the central or peripheral nervous system to reduce neuronal synchronization, or relative synchrony, without substantially affecting neuronal excitability.

In one aspect, the present invention relates to methods and agents for treating or preventing neuronal disorders, by affecting or modulating spontaneous hypersynchronized bursts of neuronal activity and the propagation of action potentials or conduction of impulses in certain cells and nerve fibers of the peripheral nervous system, for example, primary sensory afferent fibers, pain fibers, dorsal horn neurons, and supraspinal sensory and pain pathways.

The inventive treatment agents may be employed in combination with other, known, treatment agents and methods, such as those presently used in the treatment of seizure disorders, epilepsy, migraine, neuropathic pain, neuropsychiatric disorders, addictive disorders, and/or other disorders of the central and peripheral nervous systems. One of skill in the art will appreciate that the combination of a treatment agent of the present invention with another, known, treatment agent may involve both synaptic and non-synaptic mechanisms.

Treatment compositions and methods of the present invention may be used therapeutically and episodically following the onset of symptoms or prophylactically, prior to the onset of specific symptoms. For example, treatment agents of the present invention can be used to treat existing neuropathic pain or to protect nerves from neurotoxic injury and neuropathic pain secondary to chemotherapy, radiotherapy, exposure to infectious agents, and the like.

In certain embodiments, the treatment agents employed in the inventive methods are capable of crossing the blood brain barrier, and/or are administered using delivery systems that facilitate delivery of the agents to the central nervous system. For example, various blood brain barrier (BBB) permeability enhancers can be used, if desired, to transiently and reversibly increase the permeability of the blood brain barrier to a treatment agent. Such BBB permeability enhancers may include leukotrienes, bradykinin agonists, histamine, tight junction disruptors (e.g., zonulin, zot), hyperosmotic solutions (e.g., mannitol), cytoskeletal contracting agents, short chain alkylglycerols (e.g., 1-O-pentylglycerol), and others which are currently known in the art.

In a preferred embodiment, the inventive methods for treatment of a disorder of the central or peripheral nervous system involve the administration of a treatment agent comprising a diuretic (for example, a loop diuretic such as furosemide, torasemide or bumetanide, or a thiazide or thiazide-like diuretic) in combination with one or more anti-diuretic components, in order to counteract unwanted diuretic effects of the primary treatment agent. Negative side effects that can be avoided by such methods include loss of body water, and depletion of electrolytes (such as potassium, magnesium, calcium and thiamine) and B vitamins. Anti-diuretic components that may be usefully employed in such methods include, for example, antidiuretic hormones, such as vasopressin, which increases water reabsorption by the kidneys; and salts and electrolytes, which act to replenish ions lost due to diuresis. In a preferred embodiment, the diuretic treatment agent and the anti-diuretic component are combined together in a composition formulated as a liquid beverage, food or food supplement. Such compositions may also be usefully employed in the treatment of other disorders that may be effectively treated by administering diuretics, such as chronic heart failure.

Methods for screening candidate compounds for ion-dependent cotransporter agonist activity are also provided. Screening methods and systems for identifying treatment compositions of the present invention preferably employ optical, or spectroscopic, detection techniques to assess the physiological state of biological materials including cells, tissues, organs, subcellular components and intact organisms. The biological materials may be of human, animal, or plant origin, or they may be derived from any such materials. Static and dynamic changes in the geometrical structure and/or intrinsic optical properties of the biological materials in response to the administration of a physiological challenge or a test agent, are indicative and predictive of changes in the physiological state or health of the biological material. Detailed descriptions of the screening methods are provided in U.S. Pat. Nos. 6,096,510, and 6,319,682.

The above-mentioned and additional features of the present invention, together with the manner of obtaining them, will be best understood by reference to the following more detailed description. All references disclosed herein are hereby incorporated by reference in their entirety as if each was incorporated individually.

### BRIEF DESCRIPTION OF THE DRAWINGS

FIGS. 1A, 1A1, 1B, 1B1, 1C, 1C1 and 1D show the effect of furosemide on stimulation evoked after discharge activity in rat hippocampal slices.

FIGS. 2A-2R show furosemide blockade of spontaneous epileptiform burst discharges across a spectrum of in vitro models.

FIGS. 3A-3H show furosemide blockade of kainic acid-evoked electrical “status epilepticus” in urethane-anesthetized rats, with EKG recordings shown in the upper traces and cortical EEG recordings shown in the bottom traces.

FIGS. 4A and 4B show a schematic diagram of ion co-transport under conditions of reduced chloride concentration.

### DETAILED DESCRIPTION OF THE INVENTION

As discussed above, preferred treatment agents and methods of the present invention, for use in treating disorders of the central and peripheral nervous systems, modulate or disrupt the synchrony of neuronal population activity in areas of heightened synchronization by reducing the activity of NKCC co-transporters. As described in detail below and illustrated in the examples, movement of ions and modulation of ionic gradients by means of ion-dependent co-transporters, preferably cation-chloride dependent co-transporters, is critical to regulation of neuronal synchronization. Chloride co-transport function has long been thought to be directed primarily to movement of chloride out of cells. The sodium independent transporter, which has been shown to be neuronally localized, moves chloride ions out of neurons. Blockade of this transporter, such as by administration of the loop diuretic furosemide, leads to hyperexcitability, which is the short-term response to cation-chloride co-transporters such as furosemide. However, the long-term response to furosemide demonstrates that the inward, sodium-dependent movement of chloride ions, mediated by the glial associated Na⁺—K⁺—2Cl⁻ co-transporter NKCC1, plays an active role in blocking neuronal synchronization, without affecting excitability and stimulus-evoked cellular activity. Haglund and Hochman have demonstrated that the loop diuretic furosemide is able to block epileptic activity in humans while not affecting normal brain activity (J. Neurophysiol. (Feb. 23, 2005) doi:10.1 152/jn.00944.2004). These results provide support for the belief that the inventive methods and compositions may be effectively employed in the treatment of neuropathic pain without giving rise to undesirable side effects often seen with conventional treatments.

As discussed above, the NKCC1 splice variant referred to as NKCC1b is more active than the NKCC1a variant. A central or peripheral nervous system which expresses a few more percentage NKCC1b may thus be more prone to disorders such as neuropathic pain and epilepsy. Similarly, a treatment agent that is more specific for NKCC1b compared to NKCC1a may be more effective in the treatment of such disorders.

The inventive methods may be used for the treatment and/or prophylaxis of disorders of the central and peripheral nervous system, including seizures and seizure disorders, epilepsy, migraine and other headaches, cortical spreading depression, intracranial hypertension, neuropsychiatric disorders, addictive and/or compulsive disorders, the pathophysiological effects of neurotoxic agents, head trauma, stroke, ischemia and hypoxia, and neuropathic pain. In addition, the methods of the present invention may be employed to enhance certain cortical functions, such as cognitive abilities, learning and memory. Neuropathic pain having, for example, the following etiologies may be treated using the inventive compositions and methods: alcohol abuse; diabetes; eosinophilia-myalgia syndrome; Guillain-Barre syndrome; exposure to heavy metals such as arsenic, lead, mercury, and thallium; HIV/AIDS; malignant tumors; medications including amiodarone, aurothioglucose, cisplatinum, dapsone, stavudine, zalcitabine, didanosine, disulfiram, FK506, hydralazine, isoniazid, metronidazole, nitrofurantoin, paclitaxel, phenytoin and vincristine; monoclonal gammopathies; multiple sclerosis; post-stroke central pain, postherpetic neuralgia; trauma including carpal tunnel syndrome, cervical or lumbar radiculopathy, complex regional pain syndrome, spinal cord injury and stump pain; trigeminal neuralgia; vasculitis; vitamin B6 megadosing; and certain vitamin deficiencies (B12, B1, B6, E). Neuropsychiatric disorders that may be effectively treated using the inventive methods include, but are not limited to, bipolar disorders, anxiety, panic attacks, depression, schizophrenia and post-traumatic stress syndrome. Addictive and/or compulsive disorders that may be treated using the inventive compositions and methods include: eating disorders, including obesity and binge eating; alcoholism; addiction to narcotics; and smoking.

Compositions that may be effectively employed in the inventive methods are capable of reducing the effective amount, inactivating, and/or inhibiting the activity of a Na⁺—K⁺—2Cl⁻ (NKCC) co-transporter. Preferably such compositions are capable of reducing the effective amount, inactivating, and/or inhibiting the activity of the co-transporter NKCC1. In certain embodiments, the inventive compositions comprise at least one treatment agent selected from the group consisting of: antagonists of NKCC1 (including but not limited to, small molecule inhibitors of NKCC1, antibodies, or antigen-binding fragments thereof, that specifically bind to NKCC1 and soluble NKCC1 ligands); anti-sense oligonucleotides to NKCC1; NKCC1-specific small interfering RNA molecules (siRNA or RNAi); and engineered soluble NKCC1 molecules. In preferred embodiments, the treatment agent is selected from the group consisting of: loop diuretics such as furosemide, bumetanide, ethacrynic acid, torsemide, azosemide, muzolimine, piretanide, tripamide and the like; thiazide and thiazide-like diuretics, such as bendroflumethiazide, benzthiazide, chlorothiazide, hydrochlorothiazide, hydro-flumethiazide, methylclothiazide, polythiazide, trichlormethiazide, chlorthalidone, indapamide, metolazone and quinethazone; and analogs and functional derivatives of such components.

Compositions of the subject invention are suitable for human and veterinary applications and are preferably delivered as pharmaceutical compositions. Pharmaceutical compositions comprise one or more treatment agents and a physiologically acceptable carrier. Pharmaceutical compositions of the present invention may also contain other compounds, which may be biologically active or inactive. For example, one or more treatment agents of the present invention may be combined with another agent, in a treatment combination, and administered according to a treatment regimen of the present invention. Such combinations may be administered as separate compositions, combined for delivery in a complementary delivery system, or formulated in a combined composition, such as a mixture or a fusion compound. For example, in combination treatment for seizures and seizure-related disorders, such as epilepsy, treatment compositions of the present invention may be administered in combination with one or more anti-convulsants or anti-epileptic drugs. Often the dose of the anti-convulsant or anti-epileptic drug may be less than the standard dosage as a consequence of the neurophysiological activity of the inventive treatment composition. Illustrative components for use in combination with the subject compositions include, for example, phenytoin, carbamazepine, barbiturates, phenobarbital, pentobarbital, mephobarbital, trimethadione, mephenytoin, paramethadione, phenthenylate, phenacemide, metharbital, benzchlorpropanmide, phensuximide, primidone, methsuximide, ethotoin, aminoglutethimide, diazepam, clonazepam, clorazepate, fosphenytoin, ethosuximide, valporate, felbamate, gabapentin, lamotrigine, topiramate, vigrabatrin, tiagabine, zonisamide, clobazam, thiopental, midazoplam, propofol, levetiracetam, oxcarbazepine, CCPene, GYK152466 and sumatriptan. As can be readily appreciated, the above-noted compounds are only examples of suitable treatment combinations, and other compounds or similar classes of compounds are also suitable.

Additionally, the aforementioned treatment combination may include a BBB permeability enhancer and/or a hyperosmotic agent, such as hypertonic saline or mannitol. The inclusion of a hyperosmotic agent is expected to be particularly efficacious for reducing brain swelling in traumatic head injury and cerebral edema, and is also potentially useful for preventing the onset of convulsions in term infants with hypoxic-ischemic encephalopathy.

In certain embodiments, the treatment agents of the present invention comprise a diuretic, such as furosemide, or other components that lead to diuresis. In order to reduce negative side effects that may result from diuresis, such diuretic components are preferably administered in combination with an anti-diuretic component. As used herein, the term “anti-diuretic” refers to the ability to counteract unwanted side effects that accompany administration of diuretic components including, but not limited to, loss of ions and/or water. Anti-diuretic components that may be usefully employed in the inventive methods include, for example, components that suppress diuresis, such as vasopressin and desmopressin, and components which replenish water and/or ions lost due to diuresis, such as salts and electrolytes. In preferred embodiments, the anti-diuretic component provides at least one of the following: potassium ions, magnesium ions, calcium ions, sodium ions and thiamine. Magnesium, potassium, calcium and sodium ions may be provided, for example, in the form of monoaspartate hydrochloride, oxide, hydroxide, chloride, sulfate and carbonate salts. One of skill in the art will appreciate that the amount of anti-diuretic component required to effectively counteract the unwanted side effects of the diuretic component can be readily determined using art-recognized methods, such as determining the levels of electrolytes present in blood or urine samples taken before and after administration of the diuretic component.

Administration of the diuretic and the anti-diuretic component may occur either simultaneously or sequentially. The anti-diuretic component may be administered separately to the diuretic treatment agent, formulated in the same delivery system as the diuretic treatment agent, or combined with the diuretic treatment agent in, for example, a mixture or fusion compound. In a preferred embodiment, the anti-diuretic component is a mixture of sodium ions, potassium ions, and/or magnesium ions, such as those typically found in electrolyte replacement beverages, including so-called “sports drinks” and Pedialyte™, and the diuretic treatment agent and anti-diuretic component are formulated together in a liquid beverage, food or food supplement. Such liquid beverages, foods or food supplements may also contain additional, generally inactive, components such as flavorings and food colorings. One of skill in the art will appreciate that the amount of anti-diuretic component administered to a patient will vary with differing diuretic treatment agents and regimens, and from one individual to another. In general, the anti-diuretic agent will be administered in an amount sufficient to prevent the unwanted side effects caused by administration of the diuretic treatment agent alone.

While any suitable carrier known to those of ordinary skill in the art may be employed in the pharmaceutical compositions of this invention, the preferred carrier depends upon the preferred mode of administration. Compositions of the present invention may be formulated for any appropriate mode of administration, including for example, topical, oral, sublingual, nasal, inhalation (for example in either a powdered or nebulized form), rectal, intravenous (including continuous i.v. transfusion), intracranial, spinal tap, intraperitoneal, transdermal, subcutaneous or intramuscular administration. Direct intrathecal injection or administration into the cerebral spinal fluid via the spinal cord by injection, osmotic pump or other means may be employed for certain applications. The inventive compositions may also be delivered, for example injected, to or near the origin of the neuropathic pain.

For parenteral administration, such as by subcutaneous injection, the carrier preferably comprises water, saline, glycerin, propylene glycol, alcohol, a fat, a wax and/or a buffer. For oral administration, any of the above carriers, or a solid carrier such as mannitol, lactose, starch, magnesium stearate, sodium lauryl sulphate, lactose, sodium citrate, calcium carbonate, calcium phosphate, silicates, polyethylene glycol, sodium saccharine, talcum, cellulose, glucose, sucrose, dyes, and magnesium carbonate, may be employed. For rectal administration, an aqueous gel formulation, or other suitable formulations that are well known in the art may be used. Solid compositions may also be employed as fillers in soft and hard filled gelatin capsules. Preferred materials for this include lactose or mild sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For oral administration, the compositions of the present invention may be formulated as a beverage, foodstuff or food supplement. Beverage compositions that may be effectively employed in the inventive methods include, but are not limited to: milk; milk-based beverages; soft drinks (both carbonated and non-carbonated); fruit juices; vegetable juices, fruit-based beverages; vegetable-based beverages; sports beverages; fluid replacement beverages; nutritional supplement beverages; soy-based beverages; water; and teas. Alternatively the inventive compositions may be formulated as effervescent granules having a controllable rate of effervescence, as described, for example in PCT International Publication WO 01/80822, or as uniform films which dissolve rapidly on being placed in the mouth, as described in PCT International Publication no. WO 03/030883. The treatment agents described here may also be provided in the form of an aerosol for delivery by inhalation as described in U.S. Patent Application Publication No. U.S. 2004/0105815 A1.

The compositions described herein may be administered as part of a sustained release formulation. Such formulations may generally be prepared using well-known technology and administered by, for example, oral, rectal or transdermal delivery systems, or by implantation of a formulation or therapeutic device at one or more desired target site(s). Sustained-release formulations may contain a treatment composition comprising an inventive treatment agent alone, or in combination with a second treatment agent, dispersed in a carrier matrix and/or contained within a reservoir surrounded by a rate controlling membrane. Carriers for use within such formulations are biocompatible, and may also be biodegradable. According to one embodiment, the sustained release formulation provides a relatively constant level of active composition release. According to another embodiment, the sustained release formulation is contained in a device that may be actuated by the patient or medical personnel, upon onset of certain symptoms, for example, to deliver predetermined dosages of the treatment composition. The amount of the treatment composition contained within a sustained release formulation depends upon the site of implantation, the rate and expected duration of release, and the nature of the condition to be treated or prevented.

In certain embodiments, compositions of the present invention are administered using a formulation and a route of administration that facilitates delivery of the treatment composition(s) to the central nervous system. Treatment compositions, such as NKCC1 antagonists, may be formulated to facilitate crossing of the blood brain barrier as described above, or may be co-administered with an agent that crosses the blood brain barrier. Treatment compositions may be delivered in liposome formulations, for example, that cross the blood brain barrier, or may be co-administered with other compounds, such as bradykinins, bradykinin analogs or derivatives, or other compounds, such as SERAPORT™, that cross the blood brain barrier. Alternatively, treatment compositions of the present invention may be delivered using a spinal tap that places the treatment composition directly in the circulating cerebrospinal fluid. For some treatment conditions, such as chronic epilepsy and episodic seizures, and during some episodes of spreading depression and migraine headache, there may be transient or permanent breakdowns of the blood brain barrier and specialized formulation of the treatment composition to cross the blood brain barrier may not be necessary. We have determined, for example, that a bolus iv injection of 20 mg furosemide reduces or abolishes both spontaneous interictal activity and electrical stimulation-evoked epileptiform activity in human patients who are refractory to antiepileptic drugs (AEDs) (Haglund & Hochman J. Neurophysiol. (Feb. 23, 2005) doi:10.1152/jn.00944.2004).

Local intracerebral administration, which reduces systemic distribution of the treatment composition(s), may be provided by perfusion via a mechanized delivery system, such as an osmotic pump, or by implantation of a dosage of the treatment composition(s) incorporated in a non-reactive carrier to provide controlled diffusion of the treatment composition over a time course to a circumscribed region of the brain. Other types of time release formulations may also be implemented. Additionally, direct intrathecal injection or administration into the cerebral spinal fluid via the spinal cord by injection, osmotic pump or other means is preferred for certain applications.

Routes and frequency of administration of the therapeutic compositions disclosed herein, as well as dosages, vary according to the indication, and from individual to individual, and may be readily determined by a physician from information that is generally available, and by monitoring patients and adjusting the dosages and treatment regimen accordingly using standard techniques. In general, appropriate dosages and treatment regimen provide the active composition(s) in an amount sufficient to provide therapeutic and/or prophylactic benefit. Dosages and treatment regimen may be established by monitoring improved clinical outcomes in treated patients as compared to non-treated patients. A therapeutically effective dose is an amount of a compound that, when administered as described above, produces a therapeutic response in a patient. Therapeutically effective dosages and treatment regimen will depend on the condition, the severity of the condition, and the general state of the patient being treated. Since the pharmacokinetics and pharmacodynamics of the treatment compositions of the present invention vary in different patients, a preferred method for determining a therapeutically effective dosage in a patient is to gradually escalate the dosage and monitor the clinical and laboratory indicia. For combination therapy, the two or more agents are coadministered such that each of the agents is present in a therapeutically effective amount for sufficient time to produce a therapeutic or prophylactic effect. The term “coadministration” is intended to encompass simultaneous or sequential administration of two or more agents in the same formulation or unit dosage form or in separate formulations. Appropriate dosages and treatment regimen for treatment of acute episodic conditions, chronic conditions, or prophylaxis will necessarily vary to accommodate the condition of the patient.

By way of example, furosemide may be administered orally to a patient in amounts of 10-40 mg at a frequency of 1-3 times per day, preferably in an amount of 40 mg three times per day. In an alternative example, bumetanide may be administered orally for the treatment of neuropathic pain in amounts of 1-10 mg at a frequency of 1-3 times per day. One of skill in the art will appreciate that smaller doses may be employed, for example, in pediatric applications.

Methods and systems of the present invention may also be used to evaluate candidate compounds and treatment regimen for the treatment and/or prophylaxis of disorders of the central and peripheral nervous systems. Various techniques for generating candidate compounds potentially having the desired NKCC1 cotransporter antagonist activity may be employed. Candidate compounds may be generated using procedures well known to those skilled in the art of synthetic organic chemistry. Structure-activity relationships and molecular modeling techniques are useful for the purpose of modifying known NKCC1 antagonists, such as loop diuretics, including furosemide, bumetanide, ethacrinic acid and related compounds, to confer the desired activities and specificities. Methods for screening candidate compounds for desired activities are described in U.S. Pat. Nos. 5,902,732, 5,976,825, 6,096,510 and 6,319,682, which are incorporated herein by reference in their entireties.

Candidate compounds may be screened for NKCC1 antagonist activity using screening methods of the present invention with various types of cells in culture such as glial cells, neuronal cells, renal cells, and the like, or in situ in animal models. Screening techniques to identify chloride cotransporter antagonist activity, for example, may involve altering the ionic balance of the extracellular space in the tissue culture sample, or in situ in an animal model, by producing a higher than “normal” anionic chloride concentration. The geometrical and/or optical properties of the cell or tissue sample subject to this altered ionic balance are determined, and candidate agents are administered. Following administration of the candidate agents, the corresponding geometrical and/or optical properties of the cell or tissue sample are monitored to determine whether the ionic imbalance remains, or whether the cells responded by altering the ionic balances in the extracellular and intracellular space. If the ionic imbalance remains, the candidate agent is likely a chloride cotransporter antagonist. By screening using various types of cells or tissues, candidate compounds having a high level of glial cell chloride cotransporter antagonist activity and having a reduced level of neuronal cell and renal cell chloride cotransporter antagonist activity may be identified. Similarly, effects on different types of cells and tissue systems may be assessed.

Additionally, the efficacy of candidate compounds may be assessed by simulating or inducing a condition, such as neuropathic pain, in situ in an animal model, monitoring the geometrical and/or optical properties of the cell or tissue sample during stimulation of the condition, administering the candidate compound, then monitoring the geometrical and/or optical properties of the cell or tissue sample following administration of the candidate compound, and comparing the geometrical and/or optical properties of the cell or tissue sample to determine the effect of the candidate compound. Testing the efficacy of treatment compositions for relief of neuropathic pain, for example, can be carried using well known methods and animal models, such as that described in Bennett, Hosp. Pract. (Off Ed). 33:95-98, 1998.

As discussed above, compositions for use in the inventive methods may comprise a treatment agent selected from the group consisting of: antibodies, or antigen-binding fragments thereof, that specifically bind to NKCC1; soluble ligands that bind to NKCC1; anti-sense oligonucleotides to NKCC1; and small interfering RNA molecules (siRNA or RNAi) that are specific for NKCC1.

Antibodies that specifically bind to NKCC1 are known in the art and include those available from Alpha Diagnostic International, Inc. (San Antonio, Tex. 78238). An “antigen-binding site,” or “antigen-binding fragment” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

A number of molecules are known in the art that comprise antigen-binding sites capable of exhibiting the binding properties of an antibody molecule. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. An “Fv” fragment can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al. Proc. Natl. Acad. Sci. USA 69:2659-2662, 1972; Hochman et al. Biochem 15:2706-2710, 1976; and Ehrlich et al. Biochem 19:4091-4096, 1980).

Humanized antibodies that specifically bind to NKCC1 may also be employed in the inventive methods. A number of humanized antibody molecules comprising an antigen-binding site derived from a non-human immunoglobulin have been described, including chimeric antibodies having rodent V regions and their associated CDRs fused to human constant domains (Winter et al. Nature 349:293-299, 1991; Lobuglio et al. Proc. Natl. Acad. Sci. USA 86:4220-4224, 1989; Shaw et al. J. Immunol. 138:4534-4538, 1987; and Brown et al. Cancer Res. 47:3577-3583, 1987); rodent CDRs grafted into a human supporting FR prior to fusion with an appropriate human antibody constant domain (Riechmann et al. Nature 332:323-327, 1988; Verhoeyen et al. Science 239:1534-1536, 1988; and Jones et al. Nature 321:522-525, 1986); and rodent CDRs supported by recombinantly veneered rodent FRs (European Patent Publication No. 519,596, published Dec. 23, 1992). These “humanized” molecules are designed to minimize unwanted immunological responses towards rodent antihuman antibody molecules which limit the duration and effectiveness of therapeutic applications of those moieties in human recipients.

Modulating the activity of NKCC1 may alternatively be accomplished by reducing or inhibiting expression of the polypeptide, which can be achieved by interfering with transcription and/or translation of the corresponding polynucleotide. Polypeptide expression may be inhibited, for example, by introducing anti-sense expression vectors, anti-sense oligodeoxyribonucleotides, anti-sense phosphorothioate oligodeoxy-ribonucleotides, anti-sense oligoribonucleotides or anti-sense phosphorothioate oligoribonucleotides; or by other means well known in the art. All such anti-sense polynucleotides are referred to collectively herein as “anti-sense oligonucleotides”.

The anti-sense oligonucleotides for use in the inventive methods are sufficiently complementary to the NKCC1 polynucleotide to bind specifically to the polynucleotide. The sequence of an anti-sense oligonucleotide need not be 100% complementary to the of the polynucleotide in order for the anti-sense oligonucleotide to be effective in the inventive methods. Rather an anti-sense oligonucleotide is sufficiently complementary when binding of the anti-sense oligonucleotide to the polynucleotide interferes with the normal function of the polynucleotide to cause a loss of utility, and when non-specific binding of the oligonucleotide to other, non-target sequences is avoided. The design of appropriate anti-sense oligonucleotides is well known in the art. Oligonucleotides that are complementary to the 5′ end of the message, for example the 5′ untranslated sequence up to and including the AUG initiation codon, should work most efficiently at inhibiting translation. However, oligonucleotides complementary to either the 5′- or 3′-non-translated, non-coding, regions of the targeted polynucleotide may also be employed. Cell permeation and activity of anti-sense oligonucleotides can be enhanced by appropriate chemical modifications, such as the use of phenoxazine-substituted C-5 propynyl uracil oligonucleotides (Flanagan et al., Nat. Biotechnol. 17:48-52, 1999) or 2′-O-(2-methoxy) ethyl (2′-MOE)-oligonucleotides (Zhang et al., Nat. Biotechnol. 18:862-867, 2000). The use of techniques involving anti-sense oligonucleotides is well known in the art and is described, for example, in Robinson-Benion et al. (Methods in Enzymol. 254:363-375, 1995) and Kawasaki et al. (Artific. Organs 20:836-848, 1996).

Expression of the NKCC1 polypeptide may also be specifically suppressed by methods such as RNA interference (RNAi). A review of this technique is found in Science, 288:1370-1372, 2000. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression are provided in WO 99/49029, WO 99/53050 and WO01/75164, the disclosures of which are hereby incorporated by reference. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments, thereby obviating translation and expression of the gene. Additionally, an RNA-polymerase may act to facilitate the synthesis of numerous copies of the short fragments, which exponentially increases the efficiency of the system. A unique feature of RNAi is that silencing is not limited to the cells where it is initiated. The gene-silencing effects may be disseminated to other parts of an organism.

The NKCC1 polynucleotide may thus be employed to generate gene silencing constructs and/or gene-specific self-complementary, double-stranded RNA sequences that can be employed in the inventive methods using delivery methods known in the art. A gene construct may be employed to express the self-complementary RNA sequences. Alternatively, cells may be contacted with gene-specific double-stranded RNA molecules, such that the RNA molecules are internalized into the cell cytoplasm to exert a gene silencing effect. The double-stranded RNA must have sufficient homology to the NKCC1 gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.).

One RNAi technique employs genetic constructs within which sense and anti-sense sequences are placed in regions flanking an intron sequence in proper splicing orientation with donor and acceptor splicing sites. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes.

For in vivo uses, a genetic construct, anti-sense oligonucleotide or RNA molecule may be administered by various art-recognized procedures (see, e.g., Rolland, Crit. Rev. Therap. Drug Carrier Systems 15:143-198, 1998, and cited references). Both viral and non-viral delivery methods have been used for gene therapy. Useful viral vectors include, for example, adenovirus, adeno-associated virus (AAV), retrovirus, vaccinia virus and avian poxvirus. Improvements have been made in the efficiency of targeting genes to tumor cells with adenoviral vectors, for example, by coupling adenovirus to DNA-polylysine complexes and by strategies that exploit receptor-mediated endocytosis for selective targeting (see, e.g., Curiel et al., Hum. Gene Ther., 3:147-154, 1992; and Cristiano & Curiel, Cancer Gene Ther. 3:49-57, 1996). Non-viral methods for delivering polynucleotides are reviewed in Chang & Seymour, (Eds) Curr. Opin. Mol. Ther., vol. 2, 2000. These methods include contacting cells with naked DNA, cationic liposomes, or polyplexes of polynucleotides with cationic polymers and dendrimers for systemic administration (Chang & Seymour, Ibid.). Liposomes can be modified by incorporation of ligands that recognize cell-surface receptors and allow targeting to specific receptors for uptake by receptor-mediated endocytosis (see, for example, Xu et al., Mol. Genet. Metab., 64:193-197; 1998; and Xu et al., Hum. Gene Ther., 10:2941-2952, 1999).

Tumor-targeting bacteria, such as Salmonella, are potentially useful for delivering genes to tumors following systemic administration (Low et al., Nat. Biotechnol. 17:37-41, 1999). Bacteria can be engineered ex vivo to penetrate and to deliver DNA with high efficiency into, for example, mammalian epithelial cells in vivo (see, e.g., Grillot-Courvalin et al., Nat. Biotechnol. 16:862-866, 1998). Degradation-stabilized oligonucleotides may be encapsulated into liposomes and delivered to patients by injection either intravenously or directly into a target site (for example, the origin of neuropathic pain). Alternatively, retroviral or adenoviral vectors, or naked DNA expressing anti-sense RNA for the inventive polypeptides, may be administered to patients. Suitable techniques for use in such methods are well known in the art.

The present invention further contemplates a container having a combination of preselected dosages of a NKCC co-transporter antagonist, as described above, with at least one other agent selected from the group consisting of: non-steroidal anti-inflammatory drugs, neuroleptics, corticosteroids, vasoconstrictors, beta-blockers, antidepressants, anticonvulsants, particularly Depakote, Ergot alkaloids, tryptans, Acetaminophen, caffeine, Ibuprofen, Proproxyphene, oxycodone, codeine, isometheptene, serotonin receptor agonists, ergotamine, dihydroergotamine, sumatriptan, propranolol, metoprolol, atenolol, timolol, nadolol, nifeddipine, nimodipine, verapamil, aspirin, ketoprofen, tofenamic acid, mefenamic acid, naproxen, methysergide, paracetamol, clonidine, lisuride, iprazochrome, butalbital, benzodiazepines, and divalproex sodium. The combination may also comprise a BBB permeability enhancer and/or a hyperosmotic agent. The term “container” contemplates packets, jars, vials, bottles and other containers for treatment compositions in a solid or particulate delivery system, as well as syringes and other liquid containment means, such as various types of bags, vials, bottles, and the like, having contained therein preselected dosages of the combination agents of the present invention. The combination may be packaged and administered such that each composition of the combination is packaged and administered separately, or the compositions may be packaged and administered as a mixture for simultaneous administration.

The treatment compositions and methods of the present invention have been described, above, with respect to certain preferred embodiments. The Examples set forth below describe the results of specific experiments and are not intended to limit the invention in any fashion.

### EXAMPLE 1

### The Effects of Furosemide on Epileptiform Discharges in Hippocampal Slices

During these studies, spontaneous epileptiform activity was elicited by a variety of treatments. Sprague-Dawley rats (males and females; 25-35 days old) were decapitated, the top of the skull was rapidly removed, and the brain chilled with ice-cold oxygenated slicing medium. The slicing medium was a sucrose-based artificial cerebrospinal fluid (sACSF) consisting of 220 mM sucrose, 3 mM KCI, 1.25 mM NaH₂PO₄, 2 mM MgSO₄, 26 mM NaHCO₃, 2 mM CaCl₂, and 10 mM dextrose (295-305 mOsm). A hemisphere of brain containing hippocampus was blocked and glued (cyanoacrylic adhesive) to the stage of a Vibroslicer (Frederick Haer, Brunsick, Me.). Horizontal or transverse slices 400 μm thick were cut in 4° C., oxygenated (95% O₂; 5% CO₂) slicing medium. The slices were immediately transferred to a holding chamber where they remained submerged in oxygenated bathing medium (ACSF) consisting of 124 mM NaCl, 3 mM KCl, 1.25 mM NaH₂PO₄, 2 mM MgSO₄, 26 mM NaHCO₃, 2 mM CaCl₂, and 10 mM dextrose (295-305 mOsm). The slices were held at room temperature for at least 45 minutes before being transferred to a submersion-style recording chamber (all other experiments). In the recording chamber, the slices were perfused with oxygenated recording medium at 34-35° C. All animal procedures were conducted in accordance with NIH and University of Washington animal care guidelines.

In most slice experiments, simultaneous extracellular field electrode recordings were obtained from CA1 and CA3 areas. A bipolar tungsten stimulating electrode was placed on the Schaffer collaterals to evoke synaptically-driven field responses in CA1. Stimuli consisted of 100-300 μsec duration pulses at an intensity of four times the population-spike threshold. After discharges were evoked by a 2 second train of such stimuli delivered at 60 Hz. Spontaneous interictal-like bursts were observed in slices treated by the following modifications or additions to the bathing medium: 10 mM potassium (6 slices; 4 animals; average—81 bursts/min.); 200-300 μM 4-aminopyridine (4 slices; 2 animals; average—33 burst/min.); 50-100 μM bicuculline (4 slices; 3 animals; average—14 bursts/min); M Mg⁺⁺(1 hour of perfusion—3 slices; 2 animals; average—20 bursts/min. or 3 hours of perfusion—2 slices; 2 animals); zero calcium/6 mM KCI and 2 mM EGTA (4 slices; 3 animals). In all treatments, furosemide was added to the recording medium once a consistent level of bursting was established.

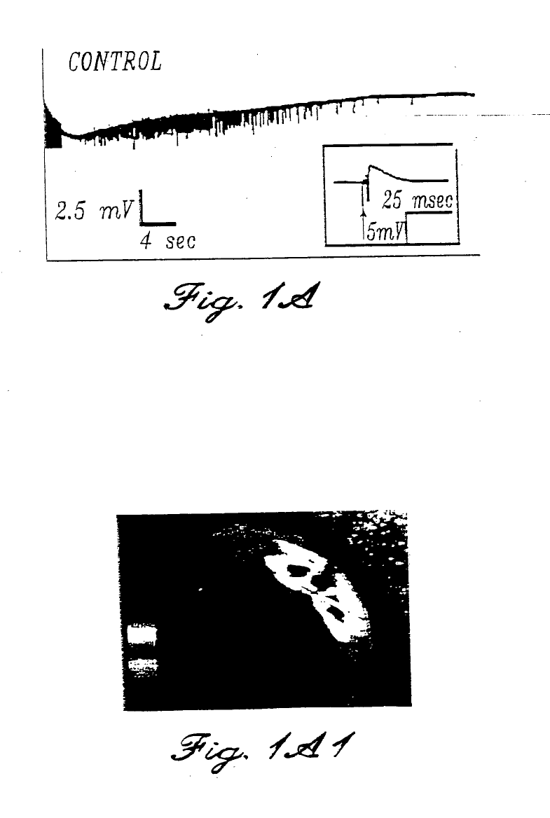

In the first of these procedures, episodes of after discharges were evoked by electrical stimulation of the Schaffer collaterals (Stasheff et al., Brain Res. 344:296, 1985) and the extracellular field response was monitored in the CA1 pyramidal cell region (13 slices; 8 animals). The concentration of Mg⁺⁺ in the bathing medium was reduced to 0.9 Mm and after discharges were evoked by stimulation at 60 Hz for 2 seconds at an intensity 4 times the population spike threshold (population spike threshold intensity varied between 20-150 μA at 100-300 μsec pulse duration). The tissue was allowed to recover for 10 minutes between stimulation trials. In each experiment, the initial response of CA1 to synaptic input was first tested by recording the field potential evoked by a single stimulus pulse. In the control condition, Schaffer collateral stimulation evoked a single population spike (FIG. 1A, inset). Tetanic stimulation evoked approximately 30 seconds after discharge (FIG. 1A, left) associated with a large change in intrinsic signal (FIG. 1A, right).

For imaging of intrinsic optical signals, the tissue was placed in a perfusion chamber located on the stage of an upright microscope and illuminated with a beam of white light (tungsten filament light and lens system; Dedo Inc.) directed through the microscope condenser. The light was controlled and regulated (power supply—Lamda Inc.) to minimize fluctuations and filtered (695 nm longpass) so that the slice was transilluminated with long wavelengths (red). Field of view and magnification were determined by the choice of microscope objectives (4× for monitoring the entire slice). Image-frames were acquired with a charge-coupled device (CCD) camera (Dage MTI Inc.) at 30 HZ and were digitized at 8 bits with a spatial resolution of 512×480 pixels using an Imaging Technology Inc. Series 151 imaging system; gains and offsets of the camera-control box and the A/D board were adjusted to optimize the sensitivity of the system. Imaging hardware was controlled by a 486-PC compatible computer. To increase signal/noise, an averaged-image was composed from 16 individual image-frames, integrated over 0.5 sec and averaged together. An experimental series typically involved the continuous acquisition of a series of averaged-images over a several minute time period; at least 10 of these averaged-images were acquired as control-images prior to stimulation. Pseudocolored images were calculated by subtracting the first control-image from subsequently acquired images and assigning a color lookup table to the pixel values. For these images, usually a linear low-pass filter was used to remove high frequency noise and a linear-histogram stretch was used to map the pixel values over the dynamic range of the system. All operations on these images were linear so that quantitative information was preserved. Noise was defined as the maximum standard deviation of fluctuations of AR/R of the sequence of control images within a given acquisition series, where AR/R represented the magnitude of the change in light-transmission through the tissue. Delta R/R was calculated by taking all the difference-images and dividing by the first control image: (subsequent image—first-control-image)/first-control-image. The noise was always <0.01 for each of the chosen image sequences. The absolute change in light transmission through the tissue was estimated during some experiments by acquiring images after placing neutral density filters between the camera and the light source. On average, the camera electronics and imaging system electronics amplified the signal 10-fold prior to digitization so that the peak absolute changes in light transmission through the tissue were usually between 1% and 2%.

The gray-scale photo shown in FIG. 1D is a video image of a typical hippocampal slice in the recording chamber. The fine gold-wire mesh that was used to hold the tissue in place can be seen as dark lines running diagonally across the slice. A stimulating electrode can be seen in the upper right on the stratum radiatum of CA1. The recording electrode (too thin to be seen in the photo) was inserted at the point indicated by the white arrow. FIG. 1A illustrates that two seconds of stimulation at 60 Hz elicited after discharge activity and shows a typical after discharge episode recorded by the extracellular electrode. The inset of FIG. 1A shows the CA1 field response to a single 200 sec test pulse (artifact at arrow) delivered to the Schaffer collaterals. FIG. 1A1 shows a map of the peak change in optical transmission through the tissue evoked by Schaffer collateral stimulation. The region of maximum optical change corresponds to the apical and basal dendritic regions of CA1 on either side of the stimulating electrode. FIG. 1B illustrates sample traces showing responses to stimulation after 20 minutes of perfusion with medium containing 2.5 mM furosemide. Both the electrical after discharge activity (shown in FIG. 1B) and the stimulation-evoked optical changes (shown in FIG. 1B1) were blocked. However, there was a hyper-excitable field response (multiple population spikes) to the test pulse (inset). FIGS. 1C and 1C1 illustrate that restoration of initial response patterns was seen after 45 minutes of perfusion with normal bathing medium.

The opposing effects of furosemide-blockade of the stimulation-evoked after discharges and a concomitant increase of the synaptic response to a test-pulse illustrate the two key results: (1) furosemide blocked epileptiform activity, and (2) synchronization (as reflected by spontaneous epileptiform activity) and excitability (as reflected by the response to a single synaptic input) were dissociated. Experiments in which the dose-dependency of furosemide was examined determined that a minimum concentration of 1.25 mM was required to block both the after discharges and optical changes.

### EXAMPLE 2

### The Effects of Furosemide on Epileptiform Discharges in Hippocampal Slices Perfused With High-K⁺ (10 mM) Bathing Medium

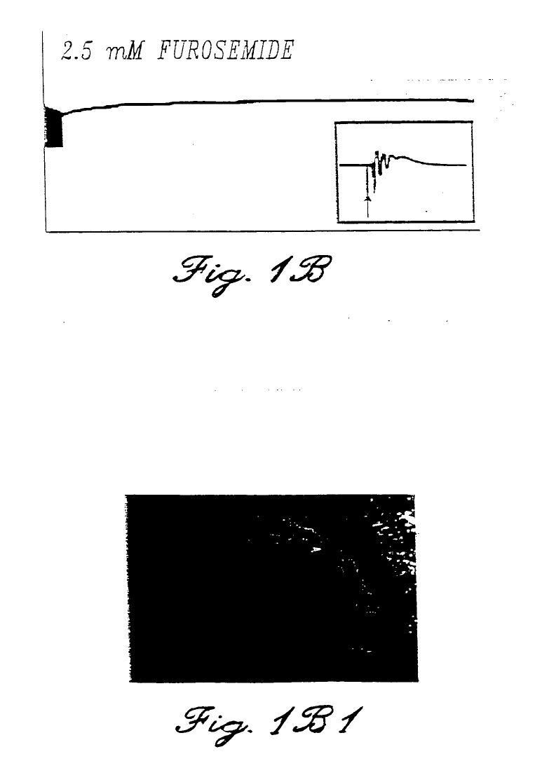

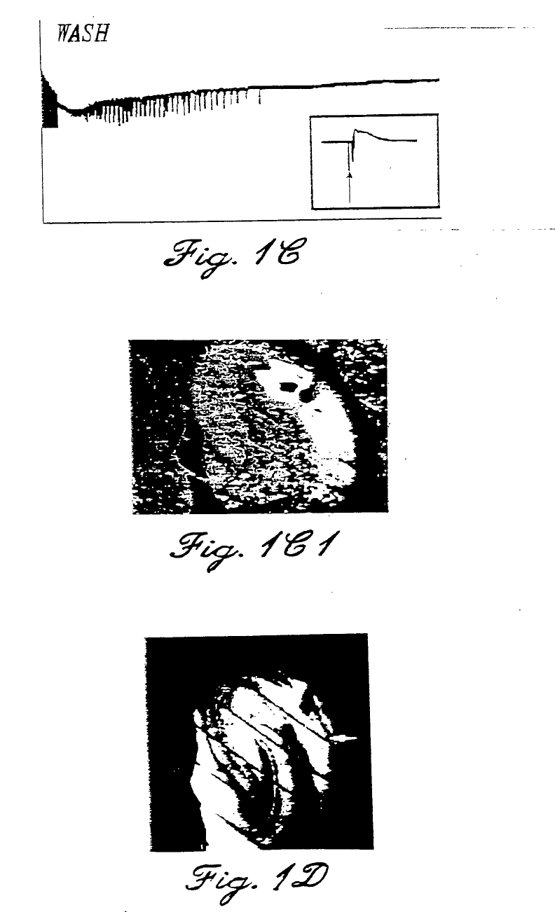

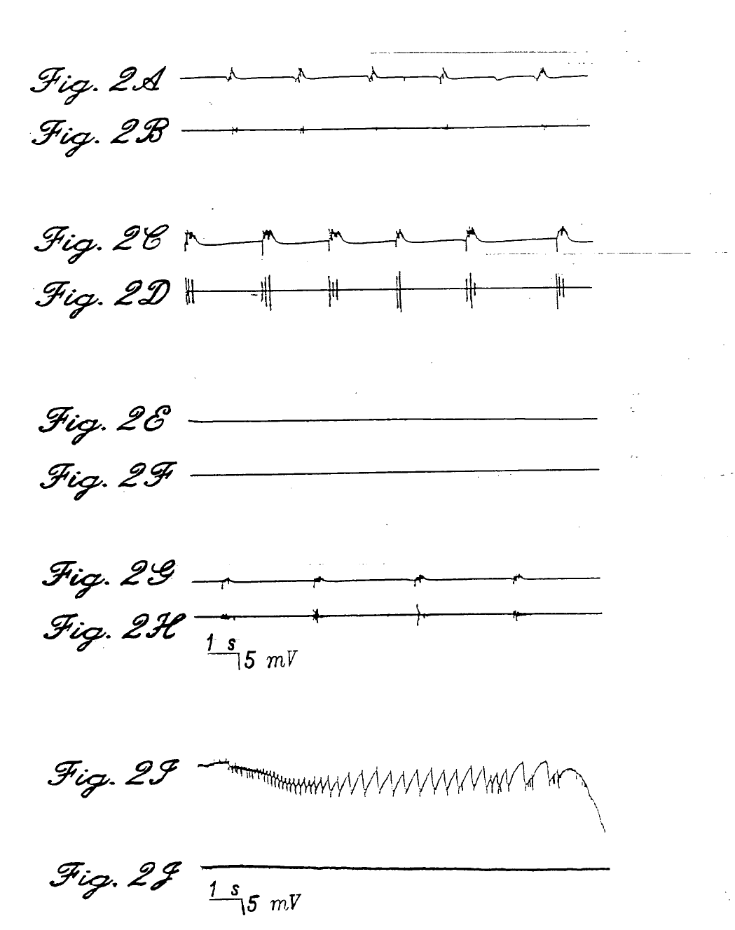

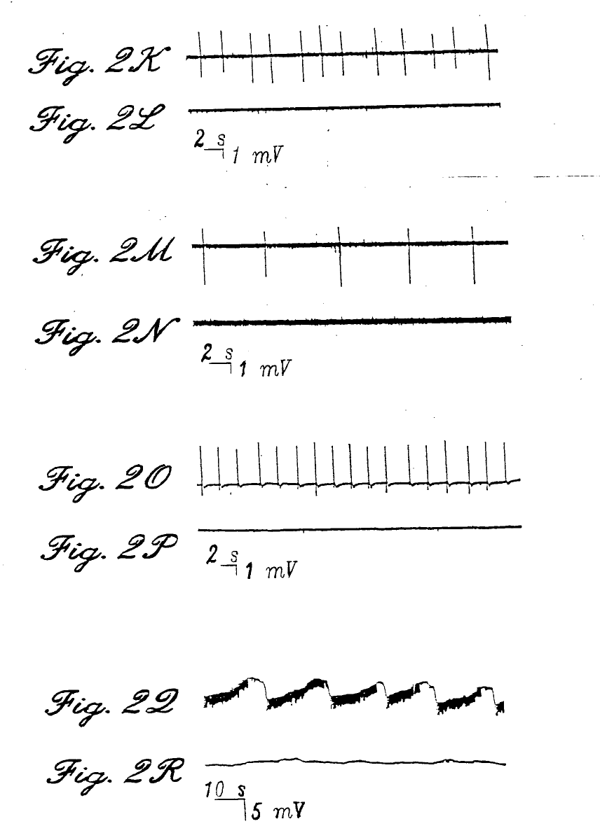

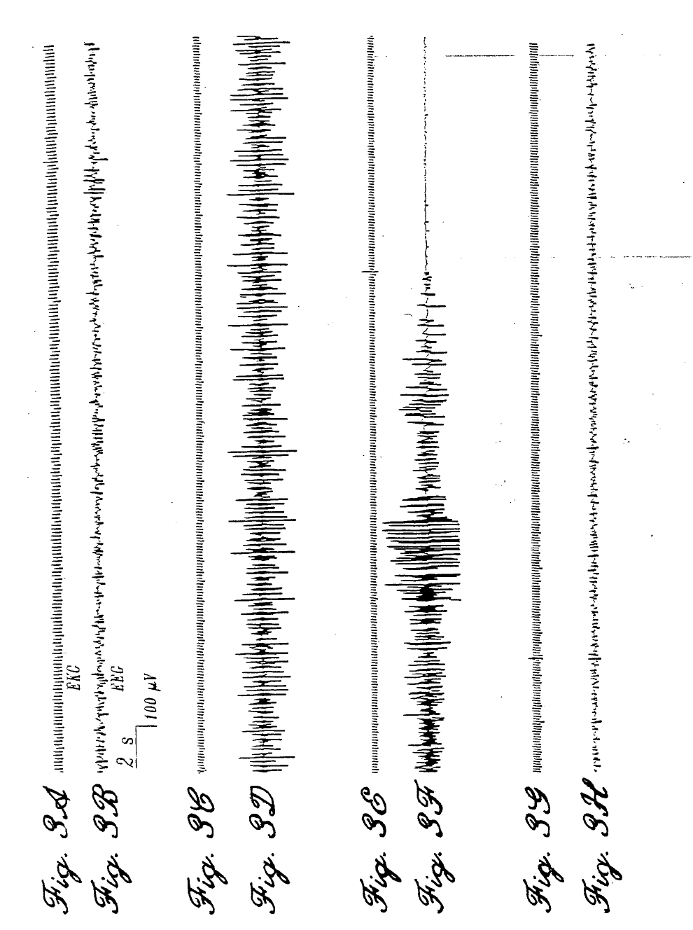

Rat hippocampal slices, prepared as described above, were perfused with a high-K⁺ solution until extended periods of spontaneous interictal-like bursting were recorded simultaneously in CA3 (top traces) and CA1 (lower traces) pyramidal cell regions (FIGS. 2A and 2B). After 15 minutes of perfusion with furosemide-containing medium (2.5 mM furosemide), the burst discharges increased in magnitude (FIGS. 2C and 2D). However, after 45 minutes of furosemide perfusion, the bursts were blocked in a reversible manner (FIGS. 2E, 2F, 2G and 2H). During this entire sequence of furosemide perfusion, the synaptic response to a single test pulse delivered to the Schaffer colalterals was either unchanged or enhanced (data not shown). It is possible that the initial increase in discharge amplitude reflected a furosemide-induced decrease in inhibition (Misgeld et al., Science 232:1413, 1986; Thompson et al., J. Neurophysiol. 60:105, 1988; Thompson and Gahwiler, J. Neuropysiol. 61:512, 1989; and Pearce, Neuron 10:189, 1993). It has previously been reported (Pearce, Neuron 10:189, 1993) that furosemide blocks a component of the inhibitory currents in hippocampal slices with a latency (<15 min.) similar to the time to onset of the increased excitability observed here. The longer latency required for the furosemide-block of the spontaneous bursting might correspond to additional time required for a sufficient block of the furosemide-sensitive cellular volume regulation mechanisms under high-K⁺ conditions.

After testing the effects of furosemide on slices perfused with high-K⁺, similar studies were performed with a variety of other commonly studied in vitro models of epileptiform discharge (Galvan et al., Brain Res. 241:75, 1982; Schwartzkroin and Prince, Brain Res. 183:61, 1980; Anderson et al., Brain Res. 398:215, 1986; and Zhang et al., Epilepsy Res. 20:105, 1995). After prolonged exposure (2-3 hours) to magnesium-free medium (0-Mg⁺⁺), slices have been shown to develop epileptiform discharges that are resistant to common clinically used anticonvulsant drugs (Zhang et al., Epilepsy Res. 20:105, 1995). Recordings from entorhinal cortex (FIG. 21) and subiculum (not shown) showed that after 3 hours of perfusion with 0-Mg⁺⁺ medium, slices developed bursting patterns that appeared similar to these previously described “anticonvulsant resistant” bursts. One hour after the addition of furosemide to the bathing medium, these bursts were blocked (FIG. 2J). Furosemide also blocked spontaneous burst discharges observed with the following additions/modifications to the bathing medium: (1) addition of 200-300 μM 4-aminopyridine (4-AP; a potassium channel blocker) (FIGS. 2K and 2L); (2) addition of the GABA antagonist, bicuculline, at 50-100 μM (FIGS. 2M and 2N); (3) removal of magnesium (0-Mg⁺⁺)—1 hours perfusion (FIGS. 20 and 2P); and (4) removal of calcium plus extracellular chelation (0-Ca⁺⁺) (FIGS. 2Q and 2R). With each of these manipulations, spontaneous interictal-like patterns were simultaneously recorded from CA1 and CA3 subfields (FIGS. 2K, 2L, 2M and 2N show only the CA3 trace and FIGS. 2O, 2P, 2Q, and 2R show only the CA1 trace). In the 0-Ca⁺⁺ experiments, 5 mM furosemide blocked the bursting with a latency of 15-20 minutes. For all other protocols, bursting was blocked by 2.5 mM furosemide with a latency of 20-60 minutes. Furosemide reversibly blocked the spontaneous bursting activity in both CA1 and CA3 in all experiments (FIGS. 2L, 2N, 2P and 2R).

### EXAMPLE 3

### The Effects of Furosemide on Epileptiform Activity Induced By i.v. Injection of Kainic Acid in Anesthetized Rats

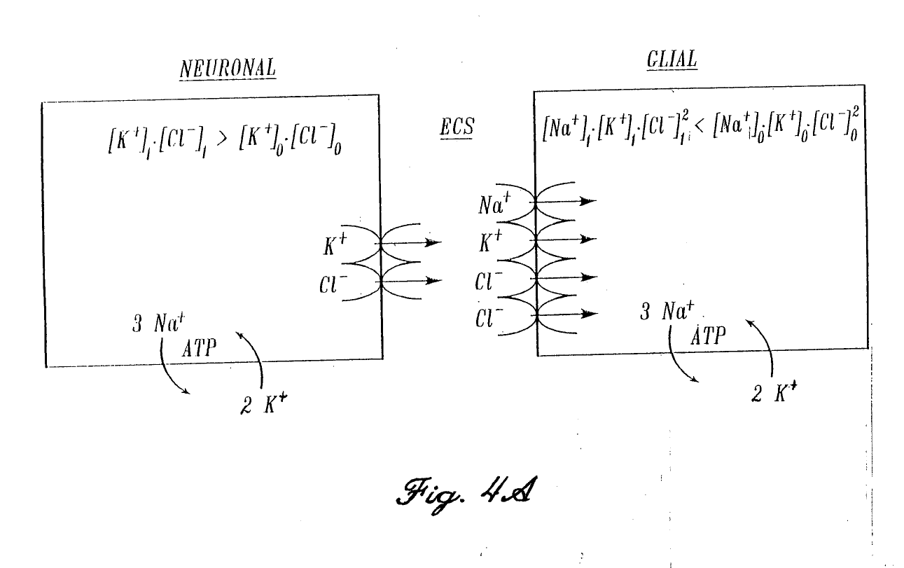

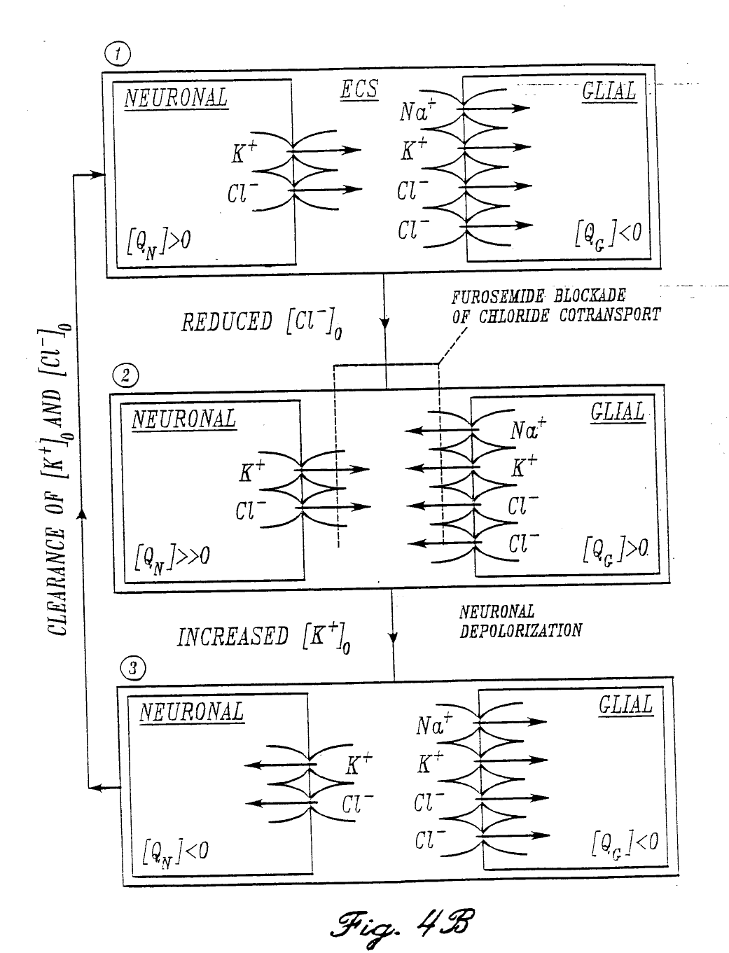

This example illustrates an in vitro model in which epileptiform activity was induced by i.v. injection of kainic acid (KA) into anesthetized rats (Lothman et al., Neurology 31:806, 1981). The results are illustrated in FIGS. 3A-3H. Sprague-Dawley rats (4 animals; weights 250-270 g) were anesthetized with urethane (1.25 g/kg i.p.) and anesthesia maintained by additional urethane injections (0.25 g/kg i.p.) as needed. Body temperature was monitored using a rectal temperature probe and maintained at 35-37° C. with a heating pad; heart rate (EKG) was continuously monitored. The jugular vein was cannulated on one side for intravenous drug administration. Rats were placed in a Kopf stereotaxic device (with the top of the skull level), and a bipolar stainless-steel microelectrode insulated to 0.5 mm of the tip was inserted to a depth of 0.5-1.2 mm from the cortical surface to record electroencephalographic (EEG) activity in the fronto-parietal cortex. In some experiments, a 2M NaCl-containing pipette was lowered to a depth of 2.5-3.0 mm to record hippocampal EEG. Data were stored on VHS videotape and analyzed off-line.

Following the surgical preparation and electrode placement, animals were allowed to recover for 30 minutes before the experiments were initiated with an injection of kainic acid (10-12 mg/kg i.v.). Intense seizure activity, an increased heart rate, and rapid movements of the vibrissae were induced with a latency of about 30 minutes. Once stable electrical seizure was evident, furosemide was delivered in 20 mg/kg boluses every 30 minutes to a total of 3 injections. Experiments were terminated with the intravenous administration of urethane. Animal care was in accordance with NIH guidelines and approved by the University of Washington Animal Care Committee.

FIGS. 3A-3H show furosemide blockade of kainic acid-evoked electrical “status epilepticus” in urethane-anesthetized rats. EKG recordings are shown as the top traces and EEG recordings are shown as the bottom traces. In this model, intense electrical discharge (electrical “status epilepticus”) was recorded from the cortex (or from depth hippocampal electrodes) 30-60 minutes after KA injection (10-12 mg/kg) (FIGS. 3C and 3D). Control experiments (and previous reports, Lothman et al., Neurology, 31:806, 1981) showed that this status-like activity was maintained for well over 3 hours. Subsequent intravenous injections of furosemide (cumulative dose: 40-60 mg/kg) blocked seizure activity with a latency of 30-45 minutes, often producing a relatively flat EEG (FIGS. 3E, 3F, 3G and 3H). Even 90 minutes after the furosemide injection, cortical activity remained near normal baseline levels (i.e., that observed prior to the KA and furosemide injections). Studies on the pharmacokinetics of furosemide in the rat indicate that the dosages used in this example were well below toxic levels (Hammarlund and Paalzow, Biopharmaceutics Drug Disposition, 3:345, 1982).

Experimental Methods for Examples 4-7

Hippocampal slices were prepared from Sprague-Dawley adult rats as described previously. Transverse hippocampal slices 100 μm thick were cut with a vibrating cutter. Slices typically contained the entire hippocampus and subiculum. After cutting, slices were stored in an oxygenated holding chamber at room temperature for at least one hour before recording. All recordings were acquired in an interface type chamber with oxygenated (95% O₂, 5% CO₂) artificial cerebral spinal fluid (ACSF) at 34°-35° C. Normal ACSF contained (in mmol/l): 124 NaCl, 3 KCl, 1.25 NaH₂PO₄, 1.2 MgSO₄, 26 NaHCO₃, 2 CaCl₂, and 10 dextrose.

Sharp-electrodes for intracellular recordings from CA1 and CA3 pyramidal cells were filled with 4 M potassium acetate. Field recordings from the CA1 and CA3 cell body layers were acquired with low-resistance glass electrodes filled with 2 M NaCl. For stimulation of the Schaffer collateral or hilar pathways, a small monopolar tungsten electrode was placed on the surface of the slice. Spontaneous and stimulation-evoked activities from field and intracellular recordings were digitized (Neurocorder, Neurodata Instruments, New York, N.Y.) and stored on videotape. AxoScope software (Axon Instruments) on a personal computer was used for off-line analysis of data.

In some experiments, normal or low-chloride medium was used containing bicuculline (20 μM), 4-amino pyridine (4-AP) (100 μM), or high-K⁺ (7.5 or 12 mM). In all experiments, low-chloride solutions (7, and 21 mM [Cl⁻]o) were prepared by equimolar replacement of NaCl with Na⁺-gluconate (Sigma). All solutions were prepared so that they had a pH of approximately 7.4 and an osmolarity of 290-300 mOsm at 35° C. and at equilibrium from carboxygenation with 95% O₂/5% CO₂.

After placement in the interface chamber, slices were superfused at approximately 1 ml/min. At this flow-rate, it took 8-10 minutes for changes in the perfusion media to be completed. All of the times reported here have taken this delay into account and have an error of approximately ±2 minutes.

### EXAMPLE 4

### Timing of Cessation of Spontaneous Epileptiform Bursting in Areas in CA1 and CA3

The relative contributions of the factors that modulate synchronized activity vary between areas CA1 and CA3. These factors include differences in the local circuitry and region-specific differences in cell packing and volume fraction of the extracellular spaces. If the anti-epileptic effects of anion or chloride-cotransport antagonism are due to a desynchronization in the timing of neuronal discharge, chloride-cotransport blockade might be expected to differentially affect areas CA1 and CA3. To test this, a series of experiments was performed to characterize differences in the timing of the blockade of spontaneous epileptiform activity in areas CA1 and CA3.

Field activity was recorded simultaneously in areas CA1 and CA3 (approximately midway between the most proximal and distal extent the CA3 region), and spontaneous bursting was induced by treatment with high-[K⁺]o (12 μM; n=12), bicuculline (20 mM; n=12), or 4-AP (100 μM; n=5). Single electrical stimuli were delivered to the Schaffer collaterals, midway between areas CA1 and CA3, every 30 seconds so that the field responses in areas CA1 and CA3 could be monitored throughout the duration of each experiment. In all experiments, at least 20 minutes of continuous spontaneous epileptiform bursting was observed prior to switching to low [Cl⁻]o (21 mM) or furosemide-containing (2.5 mM) medium.

In all cases, after 30-40 minutes exposure to furosemide or low-chloride medium, spontaneous bursting ceased in area CA1 before the bursting ceased in area CA3. The temporal sequence of events typically observed included an initial increase in burst frequency and amplitude of the spontaneous field events, then a reduction in the amplitude of the burst discharges which was more rapid in CA1 than in CA3. After CA1 became silent, CA3 continued to discharge for 5-10 minutes, until it too no longer exhibited spontaneous epileptiform events.

This temporal pattern of burst cessation was observed with all epileptiform-inducing treatments tested, regardless of whether the agent used for blockade of spontaneous bursting was furosemide or low-[Cl⁻]o medium. Throughout all stages of these experiments, stimulation of the Schaffer collaterals evoked hyperexcited field responses in both the CA1 and CA3 cell body layers. Immediately after spontaneous bursting was blocked in both areas CA1 and CA3, hyperexcited population spikes could still be evoked.

We considered the possibility that the observed cessation of bursting in CA1 prior to CA3 was an artifact of the organization of synaptic contacts between these areas relative to our choice of recording sites. It is known that the projections of the various subregions of CA3 terminate in an organized fashion in CA1; CA3 cells closer to the dentate gyrus (proximal CA3) tend to project most heavily to the distal portions of CA1 (near the subicular border), whereas CA3 projections arising from cells located more distally in CA3 terminate more heavily in portions of CA1 located closer to the CA2 border. If the cessation of bursting occurs in the different subregions of CA3 at different times, the results of the above set of experiments might arise not as a difference between CA1 and CA3, but rather as a function of variability in bursting activity across CA3 subregions. We tested this possibility in three experiments. Immediately after the spontaneous bursting ceased in CA1, we surveyed the CA3 field with a recording electrode. Recordings from several different CA3 locations (from the most proximal to the most distal portions of CA3), showed that all subregions of area CA3 were spontaneously bursting during the time that CA1 was silent.

The observation that CA3 continued to discharge spontaneously after CA1 became silent was unexpected since population discharges in CA3 are generally thought to evoke discharges in CA1 through excitatory synaptic transmission. As previously described, single-pulse stimuli delivered to the Schaffer collaterals still evoked multiple population spikes in CA1 even after the blockade of spontaneous bursting; thus, hyperexcited excitatory synaptic transmissions in CA3-to-CA1 synapse was intact. Given this maintained efficacy of synaptic transmission, and the continued spontaneous field discharges in CA3, we postulated that the loss of spontaneous bursting in CA1 was due to a decrease in synchronization of incoming excitatory drive. Further, since spontaneous epileptiform discharge in CA3 also eventually ceased, perhaps this desynchronization process occurred at different times in the two hippocampal subfields.

### EXAMPLE 5

### Effect of Chloride-cotransport Antagonism on the Synchronization of CA1 and CA3 Field Population Discharges

The observation from Example 4 suggested a temporal relationship between the exposure time to low-[Cl⁻]o or furosemide-containing medium and the characteristics of the spontaneous burst activity. Further, this relationship was different between areas CA1 and CA3. In order to better characterize the temporal relationships, we compared the occurrences of CA1 action potentials and the population spike events in the field responses of CA1 and CA3 subfields during spontaneous and stimulation-evoked burst discharge.

Intracellular recordings were obtained from CA1 pyramidal cells, with the intracellular electrode placed close (<100 μM) to the CA1 field electrode. The slice was stimulated every 20 seconds with single stimuli delivered to the Schaffer collaterals. After continuous spontaneous bursting was established for at least 20 minutes, the bathing medium was switched to bicuculline-containing low-[Cl⁻]o (21 mM) medium. After approximately 20 minutes, the burst frequency and amplitude was at its greatest. Simultaneous field and intracellular recordings during this time showed that the CA1 field and intracellular recordings were closely synchronized with the CA3 field discharges. During each spontaneous discharge, the CA3 field response preceded the CA1 discharge by several milliseconds. During stimulation-evoked events, action potential discharges of the CA1 pyramidal cell were closely synchronized to both CA3 and CA1 field discharges.

With continued exposure to low-[Cl⁻]o medium, the latency between the spontaneous discharges of areas CA1 and CA3 increased, with a maximum latency of 30-40 milliseconds occurring after 30-40 minutes exposure to the bicuculline-containing low-chloride medium. During this time, the amplitude of both the CA1 and CA3 spontaneous field discharges decreased. Stimulation-evoked discharges during this time closely mimicked the spontaneously occurring discharges in morphology and relative latency. However, the initial stimulus-evoked depolarization of the neuron (presumably, the monosynaptic EPSP) began without any significant increase in latency. The time interval during which these data were acquired corresponds to the time immediately prior to the cessation of spontaneous bursting in CA1.

After 40-50 minutes perfusion with low-[Cl⁻]o medium, the spontaneous bursts were nearly abolished in CA1 but were unaffected in CA3. Schaffer collateral stimulation during this time showed that monosynaptically-triggered responses of CA1 pyramidal cells occurred without any significant increase in latency, but that stimulation-evoked field responses were almost abolished. The time interval during which these data were acquired corresponds to the moments immediately prior to the cessation of spontaneous bursting in CA3.

After prolonged exposure to low-[Cl⁻]o medium, large increases (>30 milliseconds) developed in the latency between Schaffer collateral stimulation and the consequent CA3 field discharge. Eventually, no field responses could be evoked by Schaffer collateral stimulation in either areas CA1 and CA3. However, action potential discharge from CA1 pyramidal cells in response to Schaffer collateral stimulation could be evoked with little change in response latency. Indeed, for the entire duration of the experiments (greater than two hours), action potential discharges form CA1 pyramidal cells could be evoked at short latency by Schaffer collateral stimulation. Further, although stimulation-evoked hyperexcited discharges of CA3 were eventually blocked after prolonged exposure to low-[Cl⁻]o medium, the antidromic response in CA3 appeared to be preserved.

### EXAMPLE 6

### Effects of Chloride-cotransport Antagonism on the Synchronization of Burst Discharges in CA1 Pyramidal Cells

The foregoing data suggest the disappearance of the field responses may be due to a desynchronization of the occurrence of action potentials among neurons. That is, although synaptically-driven excitation of CA1 pyramidal cells was not preserved, action potential synchrony among the CA1 neuronal population was not sufficient to summate into a measurable DC field response. In order to test this, paired intracellular recordings of CA1 pyramidal cells were acquired simultaneously with CA1 field responses. In these experiments, both the intracellular electrodes and the field recording electrodes were placed within 200 μm of one another.

During the period of maximum spontaneous activity induced by bicuculline-containing low-[Cl⁻]o medium, recordings showed that action potentials between pairs of CA1 neurons and the CA1 field discharges were tightly synchronized both during spontaneous and stimulation-evoked discharges. After continued exposure to low-[Cl⁻]o medium, when the amplitude of the CA1 field discharge began to broaden and diminish, both spontaneous and stimulation-evoked discharges showed a desynchronization in the timing of the occurrences of action potentials between pairs of CA1 neurons, and between the action potentials and the field responses. This desynchronization was coincident with the suppression of CA1 field amplitude. By the time that spontaneous bursting in CA1 ceased, a significant increase in latency had developed between Schaffer collateral stimulation and CA1 field discharge. At this time, paired intracellular recordings showed a dramatic desynchronization in the timing of action potential discharge between pairs of neurons and between the occurrence of action potentials and the field discharges evoked by Schaffer collateral stimulation.

It is possible that the observed desynchronization of CA1 action potential discharge is due to the randomization of mechanisms necessary for synaptically-driven action potential generation, such as a disruption in the timing of synaptic release or random conduction failures at neuronal processes. If this were the case, then one would expect that the occurrence of action potentials between a given pair of neurons would vary randomly with respect to one another, from stimulation to stimulation. We tested this by comparing the patterns of action potential discharge of pairs of neurons between multiple consecutive stimuli of the Schaffer collaterals. During each stimulation event, the action potentials occurred at nearly identical times with respect to one another, and showed an almost identical burst morphology from stimulation to stimulation. We also checked to see whether the occurrence of action potentials between a given pair of neurons during spontaneous field discharges was fixed in time. The patterns of action potential discharges from a given pair of CA1 neurons was compared between consecutive spontaneous field bursts during the time when the occurrence of action potentials was clearly desynchronized. Just as in the case of stimulation-evoked action potential discharge described above, the action potentials generated during a spontaneous population discharge occurred at nearly identical times with respect to one another, and showed nearly identical burst morphology from one spontaneous discharge to the next.

### EXAMPLE 7

### Effects of Low-chloride Treatment on Spontaneous Synaptic Activity

It is possible that the anti-epileptic effects associated with chloride-cotransport antagonism are mediated by some action on transmitter release. Blockade of chloride-cotransport could alter the amount or timing of transmitter released from terminals, thus affecting neuronal synchronization. To test whether low-[Cl⁻]o exposure affected mechanisms associated with transmitter release, intracellular CA1 responses were recorded simultaneously with CA1 and CA3 field responses during a treatment which dramatically increases spontaneous synaptic release of transmitter from presynaptic terminals.

Increased spontaneous release of transmitter was induced by treatment with 4-AP (100 μM). After 40 minutes exposure to 4-AP-containing medium, spontaneous synchronized burst discharges were recorded in area CA1 and CA3. Switching to 4-AP-containing low-[Cl⁻]o medium led initially, as was shown previously, to enhanced spontaneous bursting. High-grain intracellular recordings showed that high-amplitude spontaneous synaptic activity was elicited by 4-AP treatment. Further exposure to low-chloride medium blocked spontaneous burst discharge in CA1, although CA3 continued to discharge spontaneously. At this time, CA1 intracellular recordings showed that spontaneous synaptic noise was further increased, and remained so for prolonged exposure times to 4-AP-containing low-chloride medium. These data suggest that mechanisms responsible for synaptic release from terminals are not adversely affected by low-chloride exposure in a manner that could explain the blockade of 4-AP-induced spontaneous bursting in CA1. These results also eliminate the possibility that the effects of low-[Cl⁻]o exposure are due to alterations in CA1 dendritic properties which would compromise their efficiency in conducting PSPs to the soma.

Experimental Methods for Examples 8 to 12

In all of the following experiments, [Cl⁻]o was reduced by equimolar replacement of NaCl with Na⁺-gluconate. Gluconate was used rather than other anion replacements for several reasons. First, patch-clamp studies have demonstrated that gluconate appears to be virtually impermeant to chloride channels, whereas other anions (including methylsulfate, sulfate, isethionate, and acetate) are permeable to varying degrees. Second, transport of extracellular potassium through glial NKCC1 cotransport is blocked when extracellular chloride is replaced by gluconate but is not completely blocked when replaced by isethionate. Since this furosemide-sensitive cotransporter plays a significant role in cell swelling and volume changes of the extracellular space (ECS), we wished to use the appropriate anion replacement so that the effects of our treatment would be comparable to previous furosemide experiments (Hochman et al. Science, 270:99-102, 1995; U.S. Pat. No. 5,902,732). Third, formate, acetate, and proprionate generate weak acids when employed as Cl⁻ substitutes and lead to a prompt fall in intracellular pH; gluconate remains extracellular and has not been reported to induce intracellular pH shifts. Fourth, for purposes of comparison we wished to use the same anion replacement that had been used in previous studies examining the effects of low-[Cl⁻]o on activity-evoked changes of the ECS.

There is some suggestion that certain anion-replacements might chelate calcium. Although subsequent work has failed to demonstrate any significant ability of anion-substitutes to chelate calcium, there is still some concern in the literature regarding this issue. Calcium chelation did not appear to be an issue in the following experiments, since resting membrane potentials remained normal and synaptic responses (indeed, hyperexcitable synaptic responses) could be elicited even after several hours of exposure to medium in which [Cl⁻]o had been reduced by gluconate substitution. Further, we confirmed that calcium concentration in our low-[Cl⁻]o -medium was identical to that in our control-medium by measurements made with Ca²⁺—selective microelectrodes.

Sprague-Dawley adult rats were prepared as previously described. Briefly, transverse hippocampal slices, 400 μm thick, were cut using a vibrating cutter. Slices typically contained the entire hippocampus and subiculum. After cutting, slices were stored in an oxygenated holding chamber for at least one hour prior to recording. All recordings were acquired in an interface type chamber with oxygenated (95% O₂/5% CO₂) artificial cerebral spinal fluid (ACSF) at 34°-35° C. Normal ACSF contained (in mmol/l): 124 NaCl, 3 KCl, 1.25 NaH₂PO₄, 1.2 MgSO₄, 26 NaHCO₃, 2 CaCl₂, and 10 dextrose. In some experiments, normal or low-chloride medium was used containing bicuculline (20 μM), 4-AP (100 μM), or high-K⁺ (12 mM). Low-chloride solutions (7, 16, and 21 mM [Cl⁻]o) were prepared by equimolar replacement of NaCl with Na⁺-gluconate (Sigma Chemical Co., St. Louis, Mo.). All solutions were prepared so that they had a pH of approximately 7.4 and an osmolarity of 290-300 mOsm at 35° C. and at equilibrium from carboxygenation with 95% O₂/5% CO₂.

Sharp-electrodes filled with 4 M potassium acetate were used for intracellular recordings from CA1 pyramidal cells. Field recordings from the CA1 or CA3 cell body layers were acquired with low-resistance glass electrodes filled with NaCl (2 M). For stimulation of the Schaffer collateral pathway, a small monopolar electrode was placed on the surface of the slice midway between areas CA1 and CA3. Spontaneous and stimulation-evoked activities from field and intracellular recordings were digitized (Neurocorder, Neurodata Instruments, New York, N.Y.), and stored on video tape. AxoScope software (Axon Instruments Inc.) on a PC-computer was used for off-line analyses of data.

Ion-selective microelectrodes were fabricated according to standard methods well known in the art. Double-barreled pipettes were pulled and broken to a tip diameter of approximately 3.0 μm. The reference barrel was filled with ACSF and the other barrel was sylanized and the tip back-filled with a resin selective for K⁺ (Coming 477317). The remainder of the sylanized barrel was filled with KCl (140 mM). Each barrel was led, via Ag/AgCl wires, to a high impedance dual-differential amplifier (WPI FD223). Each ion-selective microelectrode was calibrated by the use of solutions of known ionic composition and was considered suitable if it was characterized by a near-Nernstian slope response and if it remained stable throughout the duration of the experiment.

After placement in the interface chamber, slices were superfused at approximately 1 ml/minute. At this flow-rate, it took approximately 8-10 minutes for changes in perfusion media to be completed. All of the times reported here have taken this time-delay into account and have an error of approximately ±2 minutes.

### EXAMPLE 8

### Effects of Low-[Cl⁻]o on CA1 Field Recordings

Other studies have shown that prolonged exposure of cortical and hippocampal slices to low-[Cl⁻]o does not affect basic intrinsic and synaptic properties such as input resistance, resting membrane potential, depolarization-induced action-potential generation, or excitatory synaptic transmission. A previous study has also partly characterized the epileptogenic properties of low-[Cl⁻]o exposure to the CA1 area of hippocampus. The following studies were performed to observe the times of onset and possible cessation of low-[Cl⁻]o-induced hyperexcitability and hypersynchronization. Slices (n=6) were initially perfused with normal medium until stable intracellular and field recordings were established in a CA1 pyramidal cell and the CA1 cell body layer, respectively. In two experiments, the same cell was held throughout the entire length of the experiment (greater than 2 hours). In the remaining experiments (n=4), the initial intracellular recording was lost during the sequence of medium changes and additional recordings were acquired from different cells. Patterns of neuronal activity in these experiments were identical to those seen when a single cell was observed.

The field and intracellular electrodes were always placed in close proximity to one another (<200 μm). In each case, after approximately 15-20 minutes exposure to the low-[Cl⁻]o-medium (7 mM), spontaneous bursting developed, first at the cellular level, and then in the field. This spontaneous field activity, representing synchronized burst discharge in a large population of neurons, lasted from 5-10 minutes, after which time the field recording became silent. When the field first became silent, the cell continued to discharge spontaneously. This result suggests that population activity has been “desynchronized” while the ability of individual cells to discharge has not been impaired. After approximately 30 minutes exposure to low-[Cl⁻]o-medium, intracellular recording showed that cells continued to discharge spontaneously even though the field remained silent. The response of the cell to intracellular current injection at two time points demonstrated that the cell's ability to generate action potentials had not been impaired by low-[Cl⁻]o exposure. Further, electrical stimulation in CA1 stratum radiatum elicited burst discharges, indicating that a hyperexcitable state was maintained in the tissue.

### EXAMPLE 9

### Effects of Low-[Cl−]o on high-[K+]o-induced Epileptiform Activity in CA1

The previous set of experiments showed that tissue exposure to low-[Cl⁻]o medium induced a brief period of spontaneous field potential bursting which ceased within 10 minutes. If a reduction of [Cl⁻]o is indeed eventually capable of blocking spontaneous epileptiform (i.e. synchronized) bursting, then these results suggest that anti-epileptic effects would likely be observable only after this initial period of bursting activity has ceased. We therefore examined the temporal effects of low-[Cl⁻]o-treatment on high-[K⁺]o-induced bursting activity. Slices (n=12) were exposed to medium in which [K⁺]o had been increased to 12 mM, and field potentials were recorded with a field electrode in the CA1 cell body layer. Spontaneous field potential bursting was observed for at least 20 minutes, and then the slices were exposed to medium in which [K⁺]o was maintained at 12 mM, but [Cl⁻]o was reduced to 21 mM. Within 15-20 minutes after the tissue was exposed to the low-[Cl⁻]o/high-[K⁺]o-medium, the burst amplitude increased and each field event had a longer duration. After a brief period of this facilitated field activity (lasting 5-10 minutes), the bursting stopped. To test whether this blockade was reversible, after at least 10 minutes of field potential silence, we switched back to high-[K⁺]o-medium with normal [Cl⁻]o. The bursting returned within 20-40 minutes. Throughout each experiment, the CA1 field response to Schaffer collateral stimulation was monitored. The largest field responses were recorded just before the cessation of spontaneous bursting, during the period when the spontaneous bursts had the largest amplitude. Even after the blockade of spontaneous bursting, however, multiple population spikes were elicited by Schaffer collateral stimulation, indicating that synaptic transmission was intact, and that the tissue remained hyperexcitable.

In four slices, intracellular recordings from CA1 pyramidal cells were acquired along with the CA1 field recording. During the period of high-[K⁺]o-induced spontaneous bursting, hyperpolarizing current was injected into the cell so that postsynaptic potentials (PSPs) could be better observed. After low-[Cl⁻]o-blockade of spontaneous bursting, spontaneously occurring action potentials and PSPs were still observed. These observations further support the view that synaptic activity, per se, was not blocked by the low-[Cl⁻]o treatment.

### EXAMPLE 10

### Low-[Cl⁻]o—blockade of Epileptiform Activity Induced by 4-AP, high-[K⁺]o, and Bicuculline in CA1 and CA3

We next tested whether low-[Cl⁻]o treatment could block epileptiform activity in areas CA1 and CA3, which was elicited by different pharmacological treatments, as we had shown for furosemide treatment. For this set of experiments, we chose to test the effects of low-[Cl⁻]o treatment on spontaneous bursting which had been induced by high-[K⁺]o (12 mM) (n=5), 4-AP (100 μM) (n=4), and bicuculline (20 and 100 μM) (n=5). In each set of experiments, field responses were recorded simultaneously from areas CA1 and CA3, and in each case, the spontaneous epileptiform activity in both areas CA1 and CA3, was reversibly blocked within 30 minutes after [Cl⁻]o in the perfusion medium had been reduced to 21 mM. These data suggest that, like furosemide, low-[Cl⁻]o reversibly blocks spontaneous bursting in several of the most commonly studied in vitro models of epileptiform activity.

### EXAMPLE 11

### Comparison Between Low-[Cl⁻]o and Furosemide on Blockade of High-[K⁺]o-induced Epileptiform Activity

The data from the previous sets of experiments are consistent with the hypothesis that the anti-epileptic effects of both low-[Cl⁻]o and furosemide are mediated by their actions on the same physiological mechanisms. To further test this hypothesis, we compared the temporal sequence of effects of low-[Cl⁻]o (n=12) and furosemide (2.5 and 5 mM) (n=4) on high-[K⁺]o-induced bursting, as recorded with a field electrode in CA1. We found that both low-[Cl⁻]o and furosemide treatment induced a similar temporal sequence of effects: an initial brief period of increased amplitude of field activity, and then blockade (reversible) of spontaneous field activity. In both cases, electrical stimulation of the Schaffer collaterals elicited hyperexcited responses even after the spontaneous bursting had been blocked.

### EXAMPLE 12

### Consequences of Prolonged Exposure to Low-[CI⁻]o Medium With Varied [K+]o

In the preceding experiments, we monitored field activity in some slices for >1 hour after the spontaneous bursting had been blocked by low-[Cl⁻]o exposure. After such prolonged low-[Cl⁻]o exposure, spontaneous, long-lasting, depolarizing shifts developed. The morphology and frequency of these late-occurring field events appeared to be related to the extracellular potassium and chloride concentrations. Motivated by these observations, we performed a set of experiments in which we systematically varied [Cl⁻]o and [K⁺]o and observed the effects of these ion changes on the late-occurring spontaneous field events.

In our first set of experiments, slices were exposed to medium containing low-[Cl⁻]o (7 mM) and normal-[K⁺]o (3 mM) (n=6). After 50-70 minutes exposure to this medium, spontaneous events were recorded in area CA1; these events appeared as 5-10 mV negative shifts in the DC field, with the first episode lasting for 30-60 seconds. Each subsequent episode was longer than the previous one. This observation suggested that ion-homeostatic mechanisms were diminished over time as a result of the ion concentrations in the bathing medium. In some experiments (n=2) in which these negative DC field shifts had been induced, intracellular recordings from CA1 pyramidal cells were acquired simultaneously with the CA1 field recordings.

For these experiments, the intracellular and field recordings were acquired close to one another (<200 μm). Prior to each negative field shift (10-20 seconds), the neuron began to depolarize. Cellular depolarization was indicated by a decrease in resting membrane potential, an increase in spontaneous firing frequency, and a reduction of action potential amplitude. Coincident with the onset of the negative field shifts, the cells became sufficiently depolarized so that they were unable to fire spontaneous or current-elicited (not shown) action potentials. Since neuronal depolarization began 10-20 seconds prior to the field shift, it may be that a gradual increase in extracellular potassium resulted in the depolarization of a neuronal population, thus initiating these field events. Such an increase in [K⁺]o might be due to alterations of the chloride-dependent glial cotransport mechanisms that normally move potassium from extracellular to intracellular spaces. To test whether increases in [K⁺]o preceded these negative field shifts (and paralleled cellular depolarization), experiments (n=2) were performed in which a K⁺-selective microelectrode was used to record changes in [K⁺]o.

In each experiment, the K⁺-selective microelectrode and a field electrode were placed in the CA1 pyramidal layer close to one another (<200 μm), and a stimulation pulse was delivered to the Schaffer collaterals every 20 seconds so that the magnitude of the population spike could be monitored. Multiple spontaneously occurring negative field shifts were evoked by perfusion with low-[Cl⁻o] (7 mM) medium. Each event was associated with a significant increase in [K⁺]o, with the [K⁺]o increase starting several seconds prior to the onset of negative field shift. A slow 1.5-2.0 mM increase in [K⁺]o occurred over a time interval of approximately 1-2 minute seconds prior to the onset of each event. The stimulation-evoked field responses slowly increased in amplitude over time, along with the increasing [K⁺]o, until just before the negative field shift.

In a second set of experiments (n=4), [K⁺]o was increased to 12 mM and [Cl⁻]o was increased to 16 mM. After 50-90 minutes exposure to this medium, slow oscillations were recorded in area CA1. These oscillations were characterized by 5-10 mV negative DC shifts in the field potential and had a periodicity of approximately 1 cycle/40 seconds. Initially, these oscillations occurred intermittently and had an irregular morphology. Over time, these oscillations became continuous and developed a regular waveform. Upon exposure to furosemide (2.5 mM), the amplitude of the oscillations was gradually decreased and the frequency increased until the oscillations were completely blocked. Such low-[Cl]o—induced oscillations in tissue slices have not been previously reported. However, the temporal characteristics of the oscillatory events bear a striking resemblance to the low-[Cl⁻]o—induced [K⁺]o oscillations which were previously described in a purely axonal preparation.

In a third set of experiments (n=5) [Cl⁻]o was further increased to 21 mM and [K⁺]o was reduced back to 3 mM. In these experiments, single, infrequently occurring negative shifts of the field potential developed within 40-70 minutes (data not shown). These events (5-10 mV) lasting 40-60 seconds, occurred at random intervals, and maintained a relatively constant duration throughout the experiment. These events could sometimes be elicited by a single electrical stimulus delivered to the Schaffer collaterals.

Finally, in a final set of experiments (n=5), [Cl⁻]o was kept at 21 mM and [K⁺]o was raised to 12 mM. In these experiments, late-occurring spontaneous field events were not observed during the course of the experiments (2-3 hours).

### EXAMPLE 13

### Changes in [K⁺]₀ During Low-chloride Exposure

Sprague-Dawley adult rats were prepared as previously described. Transverse hippocampal slices, 400 μm thick, were cut with a vibrating cuter and stored in an oxygenated holding chamber for 1 hour before recording. A submersion-type chamber was used for K⁺-selective microelectrode recordings. Slices were perfused with oxygenated (95% O₂/5% CO₂) artificial cerebrospinal fluid (ACSF) at 34-35° C. Normal ACSF contained 10 mM dextrose, 124 mM NaCl, 3 mM KCl, 1.25 mM NaH₂PO₄, 1.2 mM MgSO₄, 26 mM NaHCO₃ and 2 mM CaCl₂. In some experiments, normal or low-chloride medium was used containing 4-aminopyridine (4-AP) at 100 μM. Low-chloride solutions (21 mM [Cl⁻]o) were prepared by equimolar replacement of NaCl with Na+-gluconate (Sigma Chemical Co.).

Field recordings from the CA1 or CA3 cell body layers were acquired with low-resistance glass electrodes filled with NaCl (2M). For stimulation of the Schaffer collateral pathway, a monopolar stainless-steel electrode was placed on the surface of the slide midway between areas CA1 and CA3. All recordings were digitized (Neurorocorder, Neurodata Instruments, New York, N.Y.) and stored on videotape.

K⁺ selective microelectrodes were fabricated according to standard methods. Briefly, the reference barrel of a double-barreled pipette was filled with ACSF, and the other barrel was sylanized and the tip back-filled with KCl with K⁺-selective resin (Corning 477317). Ion-selective microelectrodes were calibrated and considered suitable if they had a Nernstian slope response and remained stable throughout the duration of the experiment.

Exposure of hippocampal slices to low-[Cl⁻]o medium has been shown to include a temporally-dependent sequence of changes on the activity of CA1 pyramidal cells, with three characteristics phases, as described above. In brief, exposure to low-[Cl−]₀ medium results in a brief period of increased hyperexcitability and spontaneous epileptiform discharge. With further exposure to low-[Cl⁻]₀ medium, spontaneous epileptiform activity is blocked, but cellular hyperexcitability remains, and action potential firing times become less synchronized with one another. Lastly, with prolonged exposure, the action potential firing times become sufficiently desynchronized so that stimulation-evoked field responses completely disappear, yet individual cells continue to show monosynapticlly-evoked responses to Schaffer collateral stimulation. The following results demonstrate that the antiepileptic effects of furosemide on chloride-cotransport antagonism are independent of direct actions on excitatory synaptic transmission, and are a consequence of a desynchronization of population activity with our any associated decrease in excitability.

In six hippocampal slices, K⁺-selective and field microelectrodes were placed in the CA1 cell body layer, and a stimulating electrode was placed on the Schaffer collateral pathway, and single-pulse stimuli (300 μs) were delivered every 20 seconds. After stable baseline [K⁺]₀ was observed for at least 20 minutes, the perfusion was switched to low-[Cl⁻]₀ medium. Within 1-2 minutes of exposure to low-[Cl⁻]₀ medium, the field responses became hyperexcitable as the [K⁺]₀ began to rise. After approximately 4-5 minutes of exposure to low-[Cl⁻]₀ medium, the magnitude of the field response diminished until it was completely abolished. The corresponding recording of [K⁺]₀ showed that potassium began to rise immediately after exposure to low-[Cl⁻]₀ medium, and that the peak of this [K⁺]₀ rise corresponded in time to the maximally hyperexcitable CA1 field response. Coincident with the reduction of the magnitude of the field response, the [K⁺]₀ began to diminish until after 8-10 minutes exposure to low-[Cl⁻]₀ medium, it became constant for the remainder of the experiment at 1.8-2.5 mM above control levels. Four slices were switched back to control medium and allowed to fully recover. The experiment was then repeated with the K⁺-selective microelectrode placed in the stratum radiatum. A similar sequence of changes in [K⁺]₀ was observed in the dendritic layer, with the values of [K⁺]₀ being 0.2-0.3 mM less than those observed in the cell body layers.

In four hippocampal slices, the responses of stimulation-evoked changes in [K⁺]₀ between control conditions and after the CA1 field response was completely abolished by low-[Cl⁻]₀ exposure were compared. In each slice, the [K⁺]₀-selective measurements were acquired first in the cell body layer, and then after allowance for complete recovery in control medium, the experiment was repeated with the K⁺-selective electrode moved to the stratum radiatum. Each stimulation trial consisted of a 10 Hz volley delivered to the Schaffer collateral for 5 seconds. The peak rises in [K⁺]₀ were similar between control conditions an after prolonged exposure to low-[Cl⁻]₀ medium, and between the cell body and dendritic layers. However, the recovery times observed after prolonged exposure to low-[Cl⁻]₀ were significantly longer than those observed during control conditions.

These results demonstrate that the administration of furosemide resulted in increased [K⁺]₀ in the extracellular spaces. Exposure of the brain tissue to low-[Cl⁻]₀ medium immediately induced a rise in [K⁺]₀ by 1-2 mM, which remained throughout the duration of exposure, and was coincident with the initial increase in excitability and the eventual abolishment of the CA1 field response. This loss of CA1 field response during low-[Cl⁻]₀ exposure is most likely due to the desynchronization of neuronal firing times. Significantly, the stimulation-evoked increases in [K⁺]₀, in both the cell body and dendritic layers were nearly identical before and after the complete low-[Cl⁻]₀ blockade of the CA1 field response. This data suggests that comparable stimulation-evoked synaptic drive and action potential generation occurred under control conditions and after low [Cl⁻]₀ blockade of the field. Together these data demonstrate that the antiepileptic and desynchronizing effects of the chloride-cotransport antagonist, furosemide, are independent of direct actions on excitatory synaptic transmission and are a consequence of a desynchronization of population activity without decrease in excitability.

### EXAMPLE 14

### Changes in Extracellular pH During Low-chloride Exposure

Antagonists of the anion/chloride-dependent cotransporter, such as furosemide and low-[Cl−]₀, may affect extracellular pH transients that might contribute to the maintenance of synchronized population activity. Rat hippocampal brain slices were prepared as described in Example 13, except the NaHCO₃ was substituted by equimolar amount of HEPES (26 nM) and an interface-type chamber was used.

In four hippocampal brain slices continuous spontaneous bursting was elicited by exposure to medium containing 100 μM 4-AP, as described in Example 13. Field recordings were acquired simultaneously from the cell body layers in areas CA1 and CA3. A stimulus delivered every 30 seconds to the Schaffer collaterals throughout the duration of the experiments. After at least 20 minutes of continuous bursting was observed, the slices were exposed to nominally bicarbonate free, 4-AP-containing HEPES medium. There were no significant changes observed in the spontaneous or stimulation-evoked field responses resulting from prolonged exposure (0.2 hours) to HEPES medium. After the slices had been exposed for at least 2 hours to the HEPES medium, the perfusion was switched to 4-AP-containing HEPES medium in which the [Cl⁻]₀ had been reduced to 21 mM. Exposure to the low-[Cl⁻]₀ HEPES medium induced the identical sequences of events, and at the same time course, as had previously been observed with low-[Cl⁻]₀ NaHCO₃-containing medium. After complete blockade of spontaneous bursting, the perfusion medium was switched back to HEPES medium with normal [Cl⁻]₀. Within 20-40 minutes, spontaneous bursting resumed. At the time the spontaneous bursting had resumed, the slices had been perfused with nominally bicarbonate-free HEPES medium for greater than 3 hours.

This data suggests that the actions of chloride-cotransport antagonism on synchronization and excitability are independent of affects on the dynamics of extracellular pH.

FIG. 4 illustrates a schematic model of ion cotransport under conditions of reduced [Cl⁻]. FIG. 4A, left panel, shows that the chloride gradient necessary for the generation of IPSPs in neurons is maintained by efflux of ions through a furosemide-sensitive K⁺, Cl⁻ cotransporter. Under normal conditions, a high concentration of intracellular potassium (maintained by the 3Na⁺, 2K⁺-ATPase pump) serves as the driving force for the extrusion of Cl⁻ against its concentration gradient. In glial cells, as shown in the right panel of FIG. 4A, the movement of ions through the furosemide-sensitive NKCC co-transporter is from extracellular to intracellular spaces. The ion-gradients necessary for this cotransport are maintained, in part, by the “transmembrane sodium cycle”: sodium ions taken into glial cells through NKCC cotransport are continuously extruded by the 3Na⁺, 2K⁺, -ATPase pump so that a low intracellular sodium concentration is maintained. The rate and direction of ion-flux through the furosemide-dependent cotransporters are functionally proportional to their ion-product differences written as [K⁺]i×[Cl⁻]i−[K⁺]o×[Cl⁻]o) for neuronal K⁺, Cl⁻ cotransport and as [Na⁺]i×[K⁺]i×[Cl⁻]²i−[Na⁺]o×[K⁺]o×[Cl⁻]²o) for glial NKCC cotransport. The sign of these ion-product differences show the direction of ion transport with positive being from intracellular to extracellular spaces.

FIG. 4B shows a schematic phenomenological model that explains the emergence of the late-occurring spontaneous field events that arise as a result of prolonged low -[Cl⁻]o exposure. We denote the ion-product differences for neurons and glia as QN and QG, respectively. Under control conditions (1), the differences of the ion-products for neurons are such that K⁺ and Cl⁻ are cotransported from intracellular to extracellular spaces (QN>0); the differences in ion-products for glial cells are such that Na⁺, K⁺ and Cl⁻ are cotransported from the ECS to intracellular compartments (QG<0). When [Cl⁻]o is reduced (2), the ion-product differences are altered so that neuronal efflux of KCl is increased; however, the glial icon cotransport is reversed (QG>0), so that there is a net efflux of KCl and NaCl from intracellular to extracellular spaces. These changes result in buildup of extracellular potassium over time. Eventually, [K⁺]o reaches a level that induces the depolarization of neuronal populations, resulting in an even larger accumulation of [K⁺]o. This large accumulation of extracellular ions then serves to reverse the ion-product differences so that KCl is moved from extracellular to intracellular spaces (QN<0, QG<0) (3). Further clearance of the extracellular potassium eventually resets the transmembrane ion gradients to initial conditions. By cycling through this process, repetitive negative field events are generated.

### EXAMPLE 15

### Therapeutic Efficacy of Furosemide in the Alleviation of Pain Symptoms in an Animal Model of Neuropathic Pain

The ability of furosemide to alleviate pain is examined in rodents using the Chung model of neuropathic pain (see, for example, Walker et al. Mol. Med. Today 5:319-321, 1999). Sixteen adult male Long-Evans rats are used in this study. All rats receive spinal ligation of the L5 nerve as detailed below. Eight of the sixteen rats receive an injection (intravenous) of furosemide and the remaining eight receive intravenous injection of vehicle only. Pain threshold is assessed immediately using the mechanical paw withdrawal test. Differences in pain thresholds between the two groups are compared. If furosemide alleviates pain, the group with the furosemide treatment exhibits a higher pain threshold than the group that received vehicle.

Chung model of neuropathy

Spinal nerve ligation is performed under isoflourane anesthesia with animals placed in the prone position to access the left L4-L6 spinal nerves. Under magnification, approximately one-third of the transverse process is removed. The L5 spinal nerve is identified and carefully dissected free from the adjacent L4 spinal nerve and then tightly ligated using a 6-0 silk suture. The wound is treated with an antiseptic solution, the muscle layer is sutured, and the incision is closed with wound clips. Behavioral testing of mechanical paw withdrawal threshold takes place within a 3-7 day period following the incision. Briefly, animals are placed within a Plexiglas chamber (20×10.5×40.5 cm) and allowed to habituate for 15 min. The chamber is positioned on top of a mesh screen so that mechanical stimuli can be administered to the plantar surface of both hindpaws. Mechanical threshold measurements for each hindpaw are obtained using an up/down method with eight von Frey monofilaments (5, 7, 13, 26, 43, 64, 106, and 202 mN). Each trial begins with a von Frey force of 13 mN delivered to the right hindpaw for approximately 1 sec, and then the left hindpaw. If there is no withdrawal response, the next higher force is delivered. If there is a response, the next lower force is delivered. This procedure is performed until no response is made at the highest force (202 mN) or until four stimuli are administered following the initial response. The 50% paw withdrawal threshold for each paw is calculated using the following formula: [Xth]log=[vFr]log.+ky where [vFr] is the force of the last von Frey used, k=0.2268 which is the average interval (in log units) between the von Frey monofilaments, and y is a value that depends upon the pattern of withdrawal responses. If an animal does not respond to the highest von Frey hair (202 mN), then y=1.00 and the 50% mechanical paw withdrawal response for that paw is calculated to be 340.5 mN. Mechanical paw withdrawal threshold testing is performed three times and the 50% withdrawal values are averaged over the three trials to determine the mean mechanical paw withdrawal threshold for the right and left paw for each animal.

### EXAMPLE 16

### Therapeutic Efficacy of Furosemide in the Treatment of Addiction in an Animal Model of Amphetamine Sensitization

The therapeutic usefulness of furosemide in the treatment of behavior disorders is examined by measuring the ability of furosemide to reverse the symptoms of amphetamine sensitization in rats.

Amphetamine sensitization is induced in 16 animals. Following sensitization, the rats are divided into two equal groups (n=8). One group receives treatment with furosemide and the other half receives vehicle. All rats are then given a challenge injection of amphetamine. Open field motor activity is monitored. If furosemide reduces or blocks amphetamine sensitization, the group that received furosemide prior to the amphetamine challenge exhibits shorter distances and fewer total rears.

Following three days of handling, the animals receive daily intraperitoneal (i.p.) injections of 1.5 mg/kg amphetamine hydrochloride (injection volume 1.0 ml/kg) for 5 days (amphetamine-amphetamine group). Amphetamine is freshly diluted with saline (0.9%) every morning (injections performed between 10:00 and 12:00 h). The fifth day of treatment with amphetamine is followed by withdrawal for 48 h. Following the 48 hr withdrawal, eight, of the rats receive an injection of furosemide (i.v) and eight receive an injection of vehicle (i.v). The rats then receive a challenge injection of amphetamine (1.5 mg/kg) and are monitored for locomotor activity in an open field. All injections except the challenge injection are administered in the rats' home cage.

Locomotor activity is measured in an open field for 120 min following the amphetamine challenge. Total distance traveled and number of rears are automatically recorded and compared between groups using one-way analysis of variance.

### EXAMPLE 17

### Therapeutic Efficacy of Furosemide in Alleviating the Symptoms of Intense Anxiety or Post Traumatic Stress Disorder

The therapeutic usefulness of furosemide in the treatment of post traumatic stress disorder is examined by determining the ability of furosemide to alleviate intense anxiety in contextual fear conditioning in rats.

Contextual fear conditioning involves pairing an aversive event, in this case moderate foot shock, with a distinctive environment. The strength of the fear memory is assessed using freezing, a species-typical defensive reaction in rats, marked by complete immobility, except for breathing. If rats are placed into a distinctive environment and are immediately shocked they do not learn to fear the context. However, if they are allowed to explore the distinctive environment sometime before the immediate shock, they show intense anxiety and fear when placed back into the same environment. We can take advantage of this fact and, by procedurally dividing contextual fear conditioning into two phases, we can separately study effects of treatments on memory for the context (specifically a hippocampus based process) from learning the association between context and shock or experiencing the aversiveness of the shock (which depend upon emotional response circuitry including amygdala). Post traumatic stress syndrome (PTSD) in humans has been shown to be related to emotional response circuitry in the amygdala, and for this reason contextual memory conditioning is a widely accepted model for PTSD.

The experiment uses 24 rats. Each rat receives a single 5-min episode of exploration of a small, novel environment. Seventy-two hours later they are placed into the same environment and immediately receive a single, moderate foot-shock. Twenty-four hours later, 12 of the rats receive an injection (I.V) of furosemide. The remaining 12 rats receive an injection of the vehicle. Each rat is again placed into the same environment for 8-min during which time freezing is measured, as an index of Pavlovian conditioned fear.

In this experiment four identical chambers (20×20×15 cm) are used. All aspects of the timing and control of events are under microcomputer control (MedPC, MedAssociates Inc., Vermont, USA). Measurement of freezing is accomplished through an overhead video camera connected to the microcomputer and is automatically scored using a specialty piece of software, FreezeFrame. In Phase 1, rats are placed individually into the chambers for 5 minutes. Phase 2 begins 72 hr later, when again rats are placed individually into the same chambers but they receive an immediate foot shock (1 mA for 2 s). Thirty seconds later they are removed from the chambers. In Phase 3, 24 hr later, the rats are returned to the chambers for 8 min during which time we score freezing, our index of conditioning fear. Total freezing time will be analyzed in a one-way ANOVA with drug dose as the within-groups factor.

### EXAMPLE 18

### Therapeutic Efficacy of Furosemide in Alleviating Anxiety

The therapeutic efficacy of furosemide in alleviating anxiety is examined by evaluating the effects of furosemide in two tests of anxiety in rats. Furosemide is assessed first in the fear potentiated startle (FPS) paradigm, and secondly in the elevated plus maze test of anxiety.

FPS is a commonly used assessment of the therapeutic value of anxiolytic compounds in the rat. Twenty-four rats receive a 30 min period of habituation to the FPS apparatus. Twenty-four hours later, baseline startle amplitudes are collected. The rats are divided into two matched groups (n=12) based on baseline startle amplitudes. Following baseline startle amplitude collection, 20 light/shock pairings are delivered on two sessions over two consecutive days (i.e., 10 light/shock pairings per day). On the final day one group of 12 rats receives an injection (i.v.) of furosemide and the other group receives vehicle. Immediately following injections, startle amplitudes are assessed during startle alone trials and startle plus fear (light followed by startle) trials. Fear potentiated startle (light +startle amplitudes minus startle alone amplitudes) is compared between the treatment groups. If furosemide reduces anxiety in rats, then the group receiving this treatment exhibits lower fear potentiated startle than the vehicle treated rats.

Fear Potentiated Startle

Animals are trained and tested in four identical stabilimeter devices (Med-Associates). Briefly, each rat is placed in a small Plexiglas cylinder. The floor of each stabilimeter consists of four 6-mm-diameter stainless steel bars spaced 18 mm apart through which shock can be delivered. Cylinder movements result in displacement of an accelerometer where the resultant voltage is proportional to the velocity of the cage displacement. Startle amplitude is defined as the maximum accelerometer voltage that occurs during the first 0.25 sec after the startle stimulus is delivered. The analog output of the accelerometer is amplified, digitized on a scale of 0-4096 units and stored on a microcomputer. Each stabilimeter is enclosed in a ventilated, light-, and sound-attenuating box. All sound level measurements are made with a Precision Sound Level Meter. The noise of a ventilating fan attached to a sidewall of each wooden box produces an overall background noise level of 64 dB. The startle stimulus is a 50 ms burst of white noise (5 ms rise-decay time) generated by a white noise generator. The visual conditioned stimulus employed is illumination of a light bulb adjacent to the white noise source. The unconditioned stimulus was a 0.6 mA foot shock with duration of 0.5 sec, generated by four constant-current shockers located outside the chamber. The presentation and sequencing of all stimuli are under the control of the microcomputer.

FPS procedures consist of 5 days of testing; during days 1 and 2 baseline startle responses are collected, days 3 and 4 light/shock pairings are delivered, day 5 testing for fear potentiated startle is conducted.

Matching: On the first two days all rats are placed in the Plexiglas cylinders and 3 min later presented with 30 startle stimuli at a 30 sec interstimulus interval. An intensity of 105 dB is used. The mean startle amplitude across the 30 startle stimuli on the second day is used to assign rats into treatment groups with similar means.

Training: On the following 2 days, rats are placed in the Plexiglas cylinders. Each day following 3 min after entry, 10 CS-shock pairings are delivered. The shock is delivered during the last 0.5 sec of the 3.7 sec CSs at an average intertrial interval of 4 min (range, 3-5 min).

Testing: Rats are placed in the same startle boxes where they are trained and after 3 min are presented with 18 startle-eliciting stimuli (all at 105 dB). These initial startle stimuli are used to again habituate the rats to the acoustic startle stimuli. Thirty seconds after the last of these stimuli, each animal receives 60 startle stimuli with half of the stimuli presented alone (startle alone trials) and the other half presented 3.2 sec after the onset of the 3.7 sec CS (CS-startle trials). All startle stimuli are presented at a mean 30 sec interstimulus interval, randomly varying between 20 and 40 sec.

Measures: The treatment groups will be compared on the difference in startle amplitude between CS-startle and startle-alone trials (fear potentiation).

Elevated Plus Maze Design

The elevated plus maze (EPM) is commonly used to assess anxiety levels in rodents. The EPM takes advantage of the fact that when a normal rat is feeling anxious in a novel environment it will seek out and hide in enclosed spaces. A normal rat will venture out into open spaces within the new environment only when it feels less anxious. Drugs like diazepam and buspirone show anxiolytic effects in this task, and hence rats treated with such drugs spend more time within the open areas of the maze.

This experiment will employ 16 rats. Eight of the rats will receive an injection (i.v) of furosemide and eight will receive an injection of vehicle. Each rat will immediately be placed on the elevated plus maze. Time spent in the open arms of the maze are recorded and compared between treatment groups. If furosemide reduces anxiety in rat then the group that received the furosemide will spend more time in the open arms than the rats that received vehicle.

The elevated plus maze consists of two opposing open arms, 50×10 cm, crossed with two opposing enclosed arms of the same dimensions but with walls 40 cm high. Each of the four arms is connected to one side of a central square (10×10 cm) giving the apparatus a plus-sign appearance. The maze is elevated 50 cm above the floor in a normally illuminated room. The rats are placed individually on the central square of the plus maze facing an enclosed arm. The entire 3-min session is videotaped and later scored. The time spent and the number of entries into the open and closed arms, and the number of trips made to at least the midpoint down the open arms is recorded. An arm entry is defined as placement of all four paws onto the surface of the arm.

While the present invention has been described with reference to the specific embodiments thereof, it will be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

All patents and publications cited herein and PCT Application WO 00/37616, published Jun. 29, 2000, are specifically incorporated by reference herein in their entireties.

SEQ ID NO: 1-2 are set out in the attached Sequence Listing. The codes for polynucleotide and polypeptide sequences used in the attached Sequence Listing conform to WIPO Standard ST.25 (1988), Appendix 2.

I claim:

1. A method for treating or preventing a disorder of the central or peripheral nervous system in a mammalian subject, comprising administering to the subject:
(a) a first component having diuretic properties and being capable of inhibiting Na⁺—K⁺—2Cl⁻ (NKCC) co-transporter activity; and
(b) a second component having anti-diuretic properties,
wherein the second component is administered in an amount sufficient to counteract the diuretic properties of the first component.

2. The method of claim 1, wherein the disorder is selected from the group consisting of: neuropathic pain; addictive disorders; seizures; seizure disorders; epilepsy; status epilepticus; migraine headache; cortical spreading depression; headache; intracranial hypertension; central nervous system edema; neuropsychiatric disorders; neurotoxicity; head trauma; stroke; ischemia; and hypoxia.

3. The method of claim 1, wherein the first component is capable of inhibiting NKCC1 activity.

4. The method of claim 3, wherein the first component is an antagonist of NKCC1.

5. The method of claim 1, wherein the first component is selected from the group consisting of: loop diuretics; loop diuretic-like compositions; thiazide diuretics; thiazide diuretic-like compositions; and analogs and functional derivatives thereof.

6. The method of claim 5, wherein the first component is selected from the group consisting of: furosemide; bumetanide; ethacrynic acid; torsemide; azosemide; muzolimine; piretanide; tripamide; bendroflumethiazide; benzthiazide; chlorothiazide; hydrochlorothiazide; hydro-flumethiazide; methyclothiazide; polythiazide; trichlor-methiazide; chlorthalidone; indapamide; metolazone; and quinethazone.

7. The method of claim 1, wherein the second component is selected from the group consisting of: vasopressin; desmopressin; sodium ions; potassium ions; magnesium ions; calcium ions; thiamine; and combinations thereof.

8. The method of claim 1, wherein the first component and the second component are formulated together in an aqueous solution.

9. The method of claim 8, wherein the first component and the second components are administered in a formulation selected from the group consisting of: beverages; foodstuffs; and food supplements.

10. The method of claim 8, wherein the formulation further comprises at least one component selected from the group consisting of: flavorings and food colorings.

11. The method of claim 1, further comprising administering a composition selected from the group consisting of: phenytoin; carbamazepine; barbiturates; Phenobarbital; pentobarbital; mephobarbital; trimethadione; mephenytoin; paramethadione; phenthenylate; phenacemide; metharbital; benzchlorpropanmide; phensuximide; primidone; methsuximide; ethotoin; aminoglutethimide; diazepam; clonazepam; clorazepate; fosphenytoin; ethosuximide; valporate; felbamate; gabapentin; lamotrigine; topiramate; vigrabatrin; tiagabine; zonisamide; clobazam; thiopental; midazoplam; propofol; levetiracetam; oxcarbazepine; CCPene; GYK152466; sumatriptan; non-steroidal anti-inflammatory drugs; neuroleptics; corticosteroids; vasoconstrictors; beta-blockers; antidepressants; anticonvulsants; Ergot alkaloids, tryptans; Acetaminophen; caffeine; Ibuprofen; Proproxyphene; oxycodone; codeine; isometheptene; serotonin receptor agonists; ergotamine; dihydroergotamine; sumatriptan; propranolol; metoprolol; atenolol; timolol; nadolol; nifeddipine; nimodipine; verapamil; aspirin; ketoprofen; tofenamic acid; mefenamic acid; naproxen; methysergide; paracetamol; clonidine; lisuride; iprazochrome; butalbital; benzodiazepines; and divalproex sodium.

12. The method of claim 1, wherein the subject is a human.

13. The method of claim 1, additionally comprising administering an effective amount of a blood brain barrier permeability enhancer.

14. The method of claim 1, additionally comprising administering a hyperosmotic agent.

15. A composition comprising:
(a) a component having diuretic properties and being capable of inhibiting Na⁺—K⁺—2Cl⁻ (NKCC) co-transporter activity;
(b) potassium ions;
(c) magnesium ions;
(d) sodium ions; and
(e) calcium ions,
wherein the concentration of potassium ions, magnesium ions, sodium ions and calcium ions is sufficient to replace an amount of potassium ions, magnesium ions, sodium ions and calcium ions lost by a patient following administration of the composition to the patient.

16. The composition of claim 15, wherein the component having diuretic properties is effective in treating or preventing a disorder selected from the group consisting of: disorders of the central nervous system; and disorders of the peripheral nervous system.

17. The composition of claim 15, wherein the component having diuretic properties is effective in treating or preventing a disorder selected from the group consisting of: neuropathic pain; addictive disorders; seizures; seizure disorders; epilepsy; status epilepticus; migraine headache; cortical spreading depression; headache; intracranial hypertension; central nervous system edema; neuropsychiatric disorders; neurotoxicity; head trauma; stroke; ischemia; and hypoxia.

18. The composition of claim 15, wherein the component having diuretic properties is selected from the group consisting of: loop diuretics; loop diuretic-like compositions; thiazide diuretics; thiazide diuretic-like compositions; and analogs and functional derivatives thereof.

19. The composition of claim 15, wherein the component having diuretic properties is selected from the group consisting of: furosemide; bumetanide; ethacrynic acid; torsemide; azosemide; muzolimine; piretanide; tripamide; bendroflumethiazide; benzthiazide; chlorothiazide; hydrochlorothiazide; hydro-flumethiazide; methyclothiazide; polythiazide; trichlor-methiazide; chlorthalidone; indapamide; metolazone; and quinethazone.

20. The composition of claim 15, further comprising at least one component selected from the group consisting of: vasopressin; desmopressin; thiamine; and combinations thereof

21. The composition of claim 15, having a formulation selected from the group consisting of: beverages; foodstuffs; and food supplements.

22. A method for treating an addictive disorder in a mammalian subject, comprising administering an effective amount of a composition comprising a Na+K⁺2Cl co-transporter antagonist to the subject.

23. The method of claim 22, wherein the addictive disorder is selected from the group consisting of: eating disorders; addiction to narcotics; alcoholism; and smoking.

24. The method of claim 23, wherein the addictive disorder is an eating disorder selected from the group consisting of: obesity; and binge eating.

25. The method of claim 22, wherein the Na⁺K⁺2Cl co-transporter antagonist reduces or blocks hypersynchronized neuronal population discharges by non-synaptic effects.

26. The method of claim 22, wherein the Na⁺K⁺2Cl co-transporter antagonist is a NKCC1 co-transporter antagonist.

27. The method of claim 26, wherein the Na⁺K⁺2Cl co-transporter antagonist is a loop diuretic.

28. The method of claim 27, wherein the loop diuretic is selected from the group consisting of: furosemide; bumetanide; ethacrynic acid; torsemide; azosemide; muzolimine; .piretanide; tripamide; and functional analogs and derivatives thereof.

29. The method of claim 22, wherein the Na⁺K⁺2Cl co-transporter antagonist is selected from the group consisting of: thiazide; and thiazide-like diuretics.

30. The method of claim 29, wherein the Na⁺K⁺2Cl co-transporter antagonist is selected from the group consisting of: bendroflumethiazide; benzthiazide; chlorothiazide; hydrochlorothiazide; hydro-flumethiazide; methylclothiazide; polythiazide; trichlormethiazide; chlorthalidone; indapamide; metolazone; quinethazone; and functional analogs and derivatives thereof.

31. The method of claim 22, wherein the Na⁺K⁺2Cl co-transporter antagonist modulates extracellular ion composition and chloride gradients in nervous system tissue.

32. The method of claim 22, wherein the composition is delivered orally, sublingually, nasally, transdermally, intravenously or by inhalation.
